(19) United States
(12) Patent Application Publication
(10) Pub. No.: US 2021/0392934 A1
(43) Pub. Date: Dec. 23, 2021

(54) LIPID COMPOSITIONS AND USES THEREOF

(71) Applicant: ENZYMOTEC LTD., Kfar Baruch (IL)

(72) Inventors: Fabiana BAR-YOSEPH, Haifa (IL); Yael HERZOG, Gesher HaZiv (IL)

(73) Assignee: ENZYMOTEC LTD., Kfar Baruch (IL)

(21) Appl. No.: 16/082,202

(22) PCT Filed: Mar. 8, 2017
(86) PCT No.: PCT/IL2017/050297

§ 371 Date: Sep. 4, 2018

Related U.S. Application Data
(60) Provisional application No. 62/306,135, filed on Mar. 10, 2016.

(51) Int. Cl. A23L 33/12; A23L 33/00; A61K 31/23; A61K 9/00

(52) U.S. Cl. CPC A23L 33/12; A23L 33/30; A61K 9/0053; A61K 31/23; A23L 33/40

(57) ABSTRACT

The present invention refers to a method for one or both of improving growth in a subject and treating at least one growth problem in a subject. The method comprises administering to the subject an edible lipid composition which comprises a vegetable-derived fat source, wherein the fat source is a triglyceride fat source comprising triglycerides with 13-55% palmitic acid moieties out of the total fatty acids, and wherein the level of palmitic acid moieties at the sn-2 position of the glycerol backbone is at least 30% of total palmitic acid.

### FIELD OF THE INVENTION

The invention relates to lipid compositions and uses thereof.

### BACKGROUND OF THE INVENTION

Children growth is important for their normal development. Nutrition is a very important factor affecting children's growth.

Many factors may impair growth such as benign diseases, medications, etc. Later catch up (CU) growth may occur when the growth attenuating factor is removed.

Growth requires an adequate supply of nutrients acting as “building materials” or as regulatory factors.

A child's growth and development can be divided into four periods: Infancy, preschool years, middle childhood years and adolescence.

Immediately after birth, an infant normally growths very fast, requiring a bio-available source of energy and nutrients.

During the second half of the first year of life, growth is less rapid, and between ages 2-10 years, a child will continue to grow at a steady pace. A final growth spurt begins at the start of puberty, sometime between ages 9 and 15.

Growth occurs when bones increase in size. The growth plate at the end of the long bones of the limbs, is made of cartilage, which is a tough, elastic tissue. Cartilage cells in the growth plate multiply and move down the bone to produce a matrix, or tissue from which new bone is formed. These cartilage cells then die, leaving spaces. Special cells called osteoblasts, then produce bone to fill the spaces and replace the matrix. Once all the cartilage in the growth plate has been turned to bone, growth stops. This usually occurs before ages 16 to 18.

Abnormal formation and growth of cartilage and bone may lead to extremely short stature. Children with skeletal dysplasia or chondrodystrophies are short and have abnormal body proportions.

Breastfeeding is acknowledged as the natural and advisable way of supporting the healthy growth and development of infants due to its nutritional and immunological advantages [1]. Breast milk provides the most suitable diet for infant's nutritional requirements.

There are many different infant nutritional formulas that are commercially available. These infant formulas comprise a range of nutrients to meet the nutritional needs of the growing infant, and typically include lipids, carbohydrates, protein, vitamins, minerals and other nutrients helpful for optimal infant growth and development.

Some studies find difference in growth between breast fed and bottle fed infants during the first months of life [2] mainly due to differences in protein intake.

Infant growth begins in utero and continues rapidly after birth. If growth in the uterus is interrupted while a fetus is forming or developing, the condition is called intrauterine growth retardation or IUGR. Failure to grow normally in the uterus may result from a problem with the placenta, maternal smoking, infections and sometimes the cause cannot be determined.

Failure to thrive (FTT) anytime after birth, occurs frequently in infants and may be a result of many possible causes, such as nutrition or diseases, medical problems or factors in the child's environment such as abuse or neglect.

There are many medical causes of failure to thrive. These include: genetic problems such as Down syndrome; organ problems; hormone problems; damage to the brain or central nervous system, which may cause feeding difficulties in an infant; heart or lung problems, which can affect how nutrients move through the body; anemia or other blood disorders; gastrointestinal problems that interfere with nutrients absorption; chronic infections; metabolic disorders and problems during pregnancy or low birth weight.

Other factors in the child's environment that may affect growth include: emotional issues, poverty, malnutrition due to misunderstanding the appropriate diet needs or unavailability of proper diet, exposure to infections, parasites or toxins, poor eating habits, etc.

The child's nutrient needs correspond with the growth rates in mean of calories but also in type and adequacy of nutrition.

Children with poor nutrition or malnutrition may have poor growth. A balanced diet and adequate nutrients are essential for normal growth. Some parts of the world have serious problems of malnutrition and the growth of children may be affected in these areas.

Chronic diseases that may impair growth include diabetes, congenital heart disorders, sickle cell disease, chronic kidney failure, cystic fibrosis, crohn, inflammattory bowel disease, celiac and rheumatoid arthritis.

Growth plays a major role in children's well-being and is strongly influenced by the child's nutrition, health status and psychological stress as well as by multiple aspects of his or her environment. Thus, improving growth is of valuable importance.

Research has shown that growth problems in infants and young children are prevalent. Growth disorders may occur due to many factors. It can be a temporary growth slowing or a chronic growth retardation or interference where a child will not reach his growth potential.

WO 2005/036987 [3] describes enzymatically prepared fat base compositions which comprise a mixture of vegetable-derived triglycerides, their preparation and various uses thereof.

WO 2014/155373 [4] provides nutritional supplements designed for enhancing the growth, particularly the linear growth, of pre-pubertal children with a stature measure short compared to the norm. The nutritional composition comprises an energy source, arginine and a combination of micronutrients.

US 2007/0037861 [5] relates to the treatment of conditions and diseases for which growth hormone is a desirable method of treatment, using free fatty acid regulators in combination with growth hormone.

WO 1998/044917 [6] discloses a method for enhancing the growth of preterm infants involving the administration of certain long chain polyunsaturated fatty acids.

### REFERENCES

[1]. Agostoni C. et al., “Breast-feeding: A commentary by the ESPGHAN Committee on Nutrition”. J Pediatr Gastroenterol Nutr, 49, 112-25 2009.
[2]. Heinig, M. J., L. A. Nommsen, J. M. Peerson, B. Lonnerdal and K. G. Dewey. “Energy and protein intakes of breast-fed and formula fed infants during the first year of life and their association with growth velocity: the DARLING Study” Am J Clin Nutr, 58(2), 152-161, 1993.
[3]. WO 2005/036987.
[4]. WO 2014/155373.
[5]. US 2007/0037861.
[6]. WO 1998/044917.
[7]. Nilsson, O., E. A. Parker, A. Hegde, M. Chau, K. M. Barnes and J. Baron. “Gradients in bone morphogenetic protein-related gene expression across the growth plate” J Endocrinol, 193(1), 75-84, 2007.
[8]. Wang, Y., F. Middleton, J. A. Horton, L. Reichel, C. E. Farnum and T. A. Damron. “Microarray analysis of proliferative and hypertrophic growth plate zones identifies differentiation markers and signal pathways” Bone, 35(6), 1273-1293, 2004.

### SUMMARY OF THE INVENTION

The inventors of the present disclosure have surprisingly found that specific lipid compositions improve growth. The lipid compositions may also be useful in treating growth disorders in subjects such as infants. The lipid compositions may also be useful in improving one or more of growth related parameters as described herein below.

Thus, the present invention provides, in accordance with the first of its aspects, an edible lipid composition comprising a vegetable-derived fat source, wherein the fat source is a triglyceride fat source comprising triglycerides with 13-55% (at times 15-55%) palmitic acid moieties out of the total fatty acids, and wherein the level of palmitic acid moieties at the sn-2 position of the glycerol backbone is at least 30% of total palmitic acid, for use in one or both of improving growth in a subject and treating at least one growth problem in a subject.

In a further one of its aspects, the present invention provides an edible lipid composition comprising a vegetable-derived fat source, wherein the fat source is a triglyceride fat source comprising triglycerides with 13-55% (at times 15-55%) palmitic acid moieties out of the total fatty acids, and wherein the level of palmitic acid moieties at the sn-2 position of the glycerol backbone is at least 30% of total palmitic acid, for use in a method for one or both of improving growth in a subject and treating at least one growth problem in a subject.

In yet a further aspect, the present invention provides an edible lipid composition comprising a vegetable-derived fat source, wherein the fat source is a triglyceride fat source comprising triglycerides with 13-55% (at times 15-55%) palmitic acid moieties out of the total fatty acids, and wherein the level of palmitic acid moieties at the sn-2 position of the glycerol backbone is at least 30% of total palmitic acid, for one or both of improving growth in a subject and treating at least one growth problem in a subject.

Yet, in a further aspect, the present invention provides an edible lipid composition comprising a vegetable-derived fat source, wherein the fat source is a triglyceride fat source comprising triglycerides with 13-55% (at times 15-55%) palmitic acid moieties out of the total fatty acids, and wherein the level of palmitic acid moieties at the sn-2 position of the glycerol backbone is at least 30% of total palmitic acid, for use in the manufacture of a pharmaceutical, nutritional or a nutraceutical composition or a functional or medical food, for one or both of improving growth in a subject and treating at least one growth problem in a subject.

In another one of its aspects the present invention provides a method comprising administering to a subject an edible lipid composition comprising a vegetable-derived fat source, wherein the fat source is a triglyceride fat source comprising triglycerides with 13-55% (at times 15-55%) palmitic acid moieties out of the total fatty acids, and wherein the level of palmitic acid moieties at the sn-2 position of the glycerol backbone is at least 30% of total palmitic acid, the method being for one or both of improving growth in a subject and treating at least one growth problem in a subject.

In accordance with a further one of its aspects the present invention provides an edible vegetable-derived fat source, wherein the fat source is a triglyceride fat source comprising triglycerides with 13-55% (at times 15-55%) palmitic acid moieties out of the total fatty acids, and wherein the level of palmitic acid moieties at the sn-2 position of the glycerol backbone is at least 30% of total palmitic acid, for use in one or both of improving growth in a subject and treating at least one growth problem in a subject.

In a further one of its aspects, the present invention provides an edible vegetable-derived fat source, wherein the fat source is a triglyceride fat source comprising triglycerides with 13-55% (at times 15-55%) palmitic acid moieties out of the total fatty acids, and wherein the level of palmitic acid moieties at the sn-2 position of the glycerol backbone is at least 30% of total palmitic acid, for use in a method for one or both of improving growth in a subject and treating at least one growth problem in a subject.

In yet a further aspect, the present invention provides an edible vegetable-derived fat source, wherein the fat source is a triglyceride fat source comprising triglycerides with 13-55% (at times 15-55%) palmitic acid moieties out of the total fatty acids, and wherein the level of palmitic acid moieties at the sn-2 position of the glycerol backbone is at least 30% of total palmitic acid, for one or both of improving growth in a subject and treating at least one growth problem in a subject.

Yet, in a further aspect, the present invention provides an edible vegetable-derived fat source, wherein the fat source is a triglyceride fat source comprising triglycerides with 13-55% (at times 15-55%) palmitic acid moieties out of the total fatty acids, and wherein the level of palmitic acid moieties at the sn-2 position of the glycerol backbone is at least 30% of total palmitic acid, for use in the manufacture of a pharmaceutical, nutritional or a nutraceutical composition or a functional or medical food, for one or both of improving growth in a subject and treating at least one growth problem in a subject.

In another one of its aspects the present invention provides a method comprising administering to a subject an edible vegetable-derived fat source, wherein the fat source is a triglyceride fat source comprising triglycerides with 13-55% (at times 15-55%) palmitic acid moieties out of the total fatty acids, and wherein the level of palmitic acid moieties at the sn-2 position of the glycerol backbone is at least 30% of total palmitic acid, the method being for one or both of improving growth in a subject and treating at least one growth problem in a subject.

In another one of its aspects the present invention provides a food article, wherein the food article comprises the lipid composition and/or the vegetable-derived fat source in accordance with the invention, as described herein above and below, for (or for use in or for use in a method for) one or both of improving growth in a subject and treating at least one growth problem in a subject.

In yet another one of its aspects the present invention provides a food article, wherein the food article comprises the lipid composition and/or the vegetable-derived fat source in accordance with the invention, as described herein above and below, for (or for use in or for use in a method for) improving growth in a subject.

Yet in a further one of its aspects the present invention provides a food article, wherein the food article comprises the lipid composition and/or the vegetable-derived fat source in accordance with the invention, as described herein above and below, for (or for use in or for use in a method for) treating at least one growth disorders in a subject.

Yet in a further one of its aspects the present invention provides a food article, wherein the food article comprises the lipid composition and/or the vegetable-derived fat source in accordance with the invention, as described herein above and below, for (or for use in or for use in a method for) facilitating catch up growth in a subject.

In yet a further aspect, the present invention provides a commercial package comprising:

a) a lipid composition according to the invention and/or an edible vegetable-derived fat source according to the invention as herein disclosed which upon enteral administration to a subject improves growth in a subject (e.g., an infant) and/or treats at least one growth problem in a subject (e.g., an infant);
b) optionally, at least one of edible physiologically acceptable protein, carbohydrate, vitamin, mineral and active or non-active additives;
c) optionally, at least one edible physiologically acceptable carrier or diluent for carrying the constituent/s defined in a) and b);
d) means and receptacles for admixing the constituents defined in a), b) and/or c); and
e) instructions for use.

In a further one of its aspects the present invention provides a formula (e.g., an infant formula, a child formula) comprising the composition and/or the vegetable-derived fat source according to the invention for (or for use in or for use in a method for) one or both of improving growth and treating at least one growth problem in a subject (e.g., an infant, a child) administered with said formula.

In a further one of its aspects the present invention provides a formula (e.g., an infant formula, a child formula) comprising the composition and/or the vegetable-derived fat source according to the invention for (or for use in or for use in a method for) facilitating catch up growth in a subject (e.g., an infant, a child) administered with said formula.

In yet a further one of its aspects the present invention provides a formula comprising the composition and/or the vegetable-derived fat source according to the invention for (or for use in or for use in a method for) improving growth in a subject administered with the formula.

In yet a further one of its aspects the present invention provides a formula comprising the composition and/or the vegetable-derived fat source according to the invention for (or for use in or for use in a method for) treating at least one growth problem in a subject administered with the formula.

Yet, in a further one of its aspects the present invention provides an infant formula comprising the composition and/or the vegetable-derived fat source according to the invention for (or for use in or for use in a method for) improving growth in said infant.

Yet in a further one of its aspects the present invention provides an infant formula comprising the composition and/or the vegetable-derived fat source according to the invention for (or for use in or for use in a method for) treating at least one growth problem in said infant.

Yet in a further one of its aspects the present invention provides an infant formula comprising the composition and/or the vegetable-derived fat source according to the invention for (or for use in or for use in a method for) facilitating catch up growth in said infant.

In another one of its aspects the present invention provides the compositions, fat sources, formulas, methods and uses of same in one or more of increasing of bone length in a subject; increasing of growth plate [e.g., Epiphyseal growth plate (EGP)] height in a subject; increasing of growth plate (e.g., EGP) zone of proliferation in a subject; and increasing of growth plate (e.g., EGP) zone of maturation and hypertrophy in a subject.

In a further one of its aspects the present invention provides the compositions, fat sources, formulas, methods and uses of same in one or more of improvement of weight gain in a subject; increasing of Insulin like growth factor 1 (IGF-1) levels in a subject; reducing leptin levels in a subject; improving metabolic processes in a subject and reducing risk of obesity in a subject.

In another one of its aspects the present invention provides the compositions, fat sources, formulas, methods and uses as herein described and exemplified.

### DETAILED DESCRIPTION OF THE INVENTION

The invention provides a lipid composition for improving the growth of subjects, specifically infants and children, in particular subjects whose growth (e.g., growth rate) is lower compared to the accepted norms for their gender and/or age and/or weight in healthy subjects.

The inventors of the present disclosure have shown that subjects who were fed with the lipid composition according to the invention e.g., a diet comprising suitable lipid composition as described herein, demonstrated better growth which was even more significant among subjects who were defined as having growth problems.

Thus, the present invention provides in accordance with its first aspect an edible lipid composition comprising a vegetable-derived fat source, wherein the fat source is a triglyceride fat source comprising triglycerides with 13-55% palmitic acid moieties out of the total fatty acids and wherein the level of palmitic acid moieties at the sn-2 position of the glycerol backbone is at least 30% of total palmitic acid, for use in one or both of improving growth and treating at least one growth problem in a subject.

Unless otherwise indicated, the percentages given throughout the text are provided in w/w. The term “w/w” refers to a weight per weight ratio.

As used herein the terms “improved growth” or “improving growth” or any lingual variations thereof are interchangeable and relate to one or more of maintaining normal growth rate and/or elevating the final stature measure of a human subject [as accepted for gender and for age group of subjects (e.g., infants) based on growth charts from WHO (World Health Organization) or CDC (Center for Disease Control)]; enhancing/increasing growth rate; increasing final height; facilitating catch up growth; increasing bone length; increasing growth plates (e.g., EGP) height; increasing of growth plate (e.g., EGP) zone of proliferation, increasing of growth plate (e.g., EGP) zone of maturation and hypertrophy; reducing expression of genes related to growth suppression; and increasing expression of genes related to growth enhancement.

In some embodiments improved growth is envisaged as maintaining normal growth rate.

In some embodiments improved growth is envisaged as elevating the final stature measure of a human subject (as accepted for gender and for age group of subjects e.g., infants, based on growth charts from WHO or CDC).

In some embodiments improved growth is envisaged as maintaining normal growth rate and elevating the final stature measure of a human subject.

In some embodiments improved growth is envisaged as enhancing/increasing growth rate.

In some embodiments improved growth is envisaged as increasing final height.

In some embodiments improved growth is envisaged as facilitating catch up growth.

In some embodiments improved growth is envisaged as increasing bone length.

In some embodiments improved growth is envisaged as increasing growth plates (e.g., EGP) height.

In some embodiments improved growth is envisaged as increasing of growth plate (e.g., EGP) zone of proliferation.

In some embodiments improved growth is envisaged as increasing of growth plate (e.g., EGP) zone of maturation and hypertrophy.

In some embodiments improved growth is envisaged as reducing expression of genes related to growth suppression.

In some embodiments improved growth is envisaged as increasing expression of genes related to growth enhancement.

In some embodiments improved growth is envisaged as increasing growth related gene expression.

Inherited growth potential helps infants of all living beings grow to their species specific mature size and form. Yet there are many external factors which can hinder a child's growth, such as hormones, nutrition, diseases and environment (e.g., physical, emotional and social). In some embodiments the compositions, fat sources, formulas, methods and uses as herein described and exemplified may also be used to positively affect growth patterns for a long time period e.g., 1 month, 6 months, 1 year, 5 years, 10 years and thus may be used for maximizing the growth potential of a subject administered with same.

The compositions, fat sources, formulas, methods and uses as herein described and exemplified may also be utilized to improve one or more of weight gain, organ weight (e.g., liver), fat content (e.g., liver fat content), Insulin like growth factor 1 levels, leptin levels, and metabolic processes in a subject.

The compositions, fat sources, formulas, methods and uses as herein described and exemplified may also be utilized in reducing risk of obesity in a subject.

It is noted that in some embodiments according to the present invention, when referring to improvement in the growth of a subject and/or when referring to better growth of a subject it is provided in comparison to a subject administered with a lipid composition different from the lipid composition of the present invention e.g., a fat source with triglycerides with less than 13-55% palmitic acid moieties out of the total fatty acids and with level of palmitic acid moieties at the sn-2 position of the glycerol backbone lower than 30% of total palmitic acid (e.g., as exemplified in Table 7 herein below).

In some further embodiments according to the present invention, when referring to improvement in the growth of a subject and/or when referring to better growth of a subject it is provided in comparison to a subject administered with a placebo.

In some further embodiments according to the present invention, when referring to improvement in the growth of a subject and/or when referring to better growth of a subject it is provided in comparison to the growth of the subject prior to the administration of the lipid composition according to the present invention.

In yet some further embodiments according to the present invention, when referring to improvement in the growth of a subject and/or when referring to better growth of a subject it is provided in connection with a non-healthy subject that is administered with the lipid composition according to the present invention which upon administration the growth of said subject is improved and may also become comparable to the accepted norms for the subject's gender and/or age and/or weight.

Yet, in some further embodiments according to the present invention, when referring to improvement in the growth of a subject and/or when referring to better growth of a subject, the subject may be one which suffer from a disorder (which may be a growth related disorder) or may be a subject at risk of impaired growth (e.g., having insufficient food or other substances for good health and condition) and the compositions of the present invention may improve growth to an extent that growth may be comparable to that of a healthy subject at the same age and/or gender and/or weight.

In some embodiments the improvement in growth according to the present invention may be by several percentages e.g., about 1%, about 2%, about 3%, about 4%, about 5%, about 6%, about 7%, about 8%, about 9% or more e.g., about 10%, about 15%, about 20%, about 25%, about 30%, about 35%, about 40%, about 45%, about 50%, about 55%, about 60%, about 65%, about 70% and about 75%.

As used herein the term “growth problem” or any lingual variations thereof relate to one or more of growth disturbance; a growth disorder; failure to thrive; and abnormal growth related condition. The term may also encompass insufficient growth; insufficient growth rate; reduced growth rate; insufficient growth parameters; insufficient growth and/or growth rate and/or growth parameter/s compared to acceptable norms at the specific subject's age and/or gender and/or weight and/or health condition/s and the like.

In some embodiments the growth problem is growth disturbance.

In some embodiments the growth problem is a growth disorder.

In some embodiments the growth problem is failure to thrive.

In some embodiments the growth problem is an abnormal growth related condition.

In some embodiments the growth problem is an insufficient growth.

In some embodiments the growth problem is an insufficient growth velocity/rate.

In some embodiments the growth problem is reduced growth rate.

In some embodiments the growth problem is insufficient growth parameter/s.

In some embodiments the term insufficient is provided in comparison to acceptable norms at the specific subject's age and/or gender and/or weight and/or health condition/s, e.g., in comparison with accepted norms for a specific group of subjects such as infants at different ages.

Further non-limiting examples of growth problems include undernourishment; endocrine diseases (e.g. growth hormone deficiency and hypothyroidism); Turner syndrome; chronic disease that may impair growth such as diabetes; congenital heart disorders; sickle cell disease; chronic kidney failure; cystic fibrosis; crohn; inflammattory bowel disease; celiac and rheumatoid arthritis.

In some embodiments the growth problem is an endocrine disease.

In some embodiments the growth problem is Turner syndrome.

In some embodiments the growth problem is a chronic disease.

In some embodiments according to the invention the growth problem may be a side effect associated with a specific disorder and/or condition.

As used herein, the term “catch up” or any lingual variations thereof is to be envisaged as, but not limited to, growth rate higher than acceptable norms at the specific subject's gender and/or age and/or weight and/or health condition and the like, during a specific time period and after a period of growth retardation or growth delay.

As used herein, the term “facilitating catch up growth” or any lingual variations thereof is to be envisaged as any one of, but not limited to, initiating, activating, assisting in, increasing and enhancing catch up growth.

As used herein, the term “maintaining normal growth rate” or any lingual variations thereof is to be envisaged as any one of, but not limited to, maintaining growth rate which is similar to acceptable norms of a healthy subject of the same gender, at the same age and at times of a similar weight.

As used herein, the term “final height” or any lingual variations thereof is to be envisaged as the peak of height of a subject at the end of the normal growth period.

In a further aspect, the present invention provides an edible lipid composition comprising a vegetable-derived fat source, wherein the fat source is a triglyceride fat source comprising triglycerides with 13-55% palmitic acid moieties out of the total fatty acids, and wherein the level of palmitic acid moieties at the sn-2 position of the glycerol backbone is at least 30% of total palmitic acid, for use in a method for one or both of improving growth and treating at least one growth problem in a subject.

In yet a further aspect, the present invention provides an edible lipid composition comprising a vegetable-derived fat source, wherein the fat source is a triglyceride fat source comprising triglycerides with 13-55% palmitic acid moieties out of the total fatty acids and wherein the level of palmitic acid moieties at the sn-2 position of the glycerol backbone is at least 30% of total palmitic acid, for one or both of improving growth and treating at least one growth problem in a subject.

In yet another of its aspects, the present invention provides an edible lipid composition comprising a vegetable-derived fat source, wherein the fat source is a triglyceride fat source comprising triglycerides with 13-55% palmitic acid moieties out of the total fatty acids and wherein the level of palmitic acid moieties at the sn-2 position of the glycerol backbone is at least 30% of total palmitic acid, for use in the manufacture of a pharmaceutical, nutritional or nutraceutical composition, or a functional or medical food, for use in one or both of improving growth and treating at least one growth problem in a subject.

In a further aspect the invention provides a method comprising administering to a subject an edible lipid composition comprising a vegetable-derived fat source, wherein the fat source is a triglyceride fat source comprising triglycerides with 13-55% palmitic acid moieties out of the total fatty acids and wherein the level of palmitic acid moieties at the sn-2 position of the glycerol backbone is at least 30% of total palmitic acid, the method being for one or both of improving growth and treating at least one growth problem in a subject.

In accordance with a further one of its aspects the present invention provides an edible vegetable-derived fat source, wherein the fat source is a triglyceride fat source comprising triglycerides with 13-55% (at times 15-55%) palmitic acid moieties out of the total fatty acids, and wherein the level of palmitic acid moieties at the sn-2 position of the glycerol backbone is at least 30% of total palmitic acid, for use in one or both of improving growth in a subject and treating at least one growth problem in a subject.

In a further one of its aspects, the present invention provides an edible vegetable-derived fat source, wherein the fat source is a triglyceride fat source comprising triglycerides with 13-55% (at times 15-55%) palmitic acid moieties out of the total fatty acids, and wherein the level of palmitic acid moieties at the sn-2 position of the glycerol backbone is at least 30% of total palmitic acid, for use in a method for one or both of improving growth in a subject and treating at least one growth problem in a subject.

In yet a further aspect, the present invention provides an edible vegetable-derived fat source, wherein the fat source is a triglyceride fat source comprising triglycerides with 13-55% (at times 15-55%) palmitic acid moieties out of the total fatty acids, and wherein the level of palmitic acid moieties at the sn-2 position of the glycerol backbone is at least 30% of total palmitic acid, for one or both of improving growth in a subject and treating at least one growth problem in a subject.

Yet, in a further aspect, the present invention provides an edible vegetable-derived fat source, wherein the fat source is a triglyceride fat source comprising triglycerides with 13-55% (at times 15-55%) palmitic acid moieties out of the total fatty acids, and wherein the level of palmitic acid moieties at the sn-2 position of the glycerol backbone is at least 30% of total palmitic acid, for use in the manufacture of a pharmaceutical, nutritional or a nutraceutical composition or a functional or medical food, for one or both of improving growth in a subject and treating at least one growth problem in a subject.

Yet in a further one of its aspects the present invention provides an edible lipid composition and/or a vegetable-derived fat source in accordance with the invention, as described herein above and below, for (or for use in or for use in a method for) improvement of weight gain in a subject, e.g., during catch up growth.

Yet in a further one of its aspects the present invention provides an edible lipid composition and/or a vegetable-derived fat source in accordance with the invention, as described herein above and below, for (or for use in or for use in a method for) increasing of bone length in a subject, e.g., during catch up growth.

Yet in a further one of its aspects the present invention provides an edible lipid composition and/or a vegetable-derived fat source in accordance with the invention, as described herein above and below, for (or for use in or for use in a method for) increasing of growth plate (EGP) height in a subject, e.g., during catch up growth.

Yet in a further one of its aspects the present invention provides an edible lipid composition and/or a vegetable-derived fat source in accordance with the invention, as described herein above and below, for (or for use in or for use in a method for) increasing of growth plate (EGP) zone of proliferation in a subject, e.g., during catch up growth.

Yet in a further one of its aspects the present invention provides an edible lipid composition and/or a vegetable-derived fat source in accordance with the invention, as described herein above and below, for (or for use in or for use in a method for) increasing of growth plate (EGP) zone of maturation and hypertrophy in a subject, e.g., during catch up growth.

Yet in a further one of its aspects the present invention provides an edible lipid composition and/or a vegetable-derived fat source in accordance with the invention, as described herein above and below, for (or for use in or for use in a method for) increasing of Insulin like growth factor 1 (IGF-1) levels in a subject, e.g., during catch up growth.

Yet in a further one of its aspects the present invention provides an edible lipid composition and/or a vegetable-derived fat source in accordance with the invention, as described herein above and below, for (or for use in or for use in a method for) reducing leptin levels in a subject, e.g., during catch up growth.

Yet in a further one of its aspects the present invention provides an edible lipid composition and/or a vegetable-derived fat source in accordance with the invention, as described herein above and below, for (or for use in or for use in a method for) improving metabolic processes in a subject, e.g., during catch up growth.

Yet in a further one of its aspects the present invention provides an edible lipid composition and/or a vegetable-derived fat source in accordance with the invention, as described herein above and below, for (or for use in or for use in a method for) reducing risk of obesity in a subject, e.g., during catch up growth.

In another one of its aspects the present invention provides a method comprising administering to a subject an edible vegetable-derived fat source, wherein the fat source is a triglyceride fat source comprising triglycerides with 13-55% (at times 15-55%) palmitic acid moieties out of the total fatty acids, and wherein the level of palmitic acid moieties at the sn-2 position of the glycerol backbone is at least 30% of total palmitic acid, the method being for one or both of improving growth in a subject and treating at least one growth problem in a subject.

In another one of its aspect the present invention provides a method comprising administering to a subject an edible vegetable-derived fat source, wherein the fat source is a triglyceride fat source comprising triglycerides with 13-55% (at times 15-55%) palmitic acid moieties out of the total fatty acids, and wherein the level of palmitic acid moieties at the sn-2 position of the glycerol backbone is at least 30% of total palmitic acid, the method being for one or more of increasing of bone length in a subject; increasing of growth plate (e.g., EGP) height in a subject; increasing of growth plate (e.g., EGP) zone of proliferation in a subject and increasing of growth plate (EGP) zone of maturation and hypertrophy in a subject, e.g., during a catch up growth.

In another one of its aspect the present invention provides a method comprising administering to a subject an edible vegetable-derived fat source, wherein the fat source is a triglyceride fat source comprising triglycerides with 13-55% (at times 15-55%) palmitic acid moieties out of the total fatty acids, and wherein the level of palmitic acid moieties at the sn-2 position of the glycerol backbone is at least 30% of total palmitic acid, the method being for one or more of improving of weight gain in a subject; increasing of Insulin like growth factor 1 levels in a subject; reducing leptin levels in a subject; improving metabolic processes in a subject and reducing risk of obesity in a subject e.g., during a catch up growth.

In another one of its aspects the present invention provides the compositions, fat sources, formulas, methods and uses of same for improving metabolic processes in a subject. The improvement may or may not be related to the subject's growth.

In another one of its aspects the present invention provides the compositions, fat sources, formulas, methods and uses of same for reducing risk of obesity in a subject. The reduction may or may not be related to the subject's growth.

In another one of its aspects the present invention provides a food article, wherein the food article comprises the lipid composition and/or the vegetable-derived fat source in accordance with the invention, as described herein above and below, for (or for use in or for use in a method for) one or both of improving growth and treating at least one growth problem in a subject.

In yet another one of its aspects the present invention provides a food article, wherein the food article comprises the lipid composition and/or the vegetable-derived fat source in accordance with the invention, as described herein above and below, for (or for use in or for use in a method for) improving growth in a subject.

Yet in a further one of its aspects the present invention provides a food article, wherein the food article comprises the lipid composition and/or the vegetable-derived fat source in accordance with the invention, as described herein above and below, for (or for use in or for use in a method for) treating at least one growth disorders in a subject.

Yet in a further one of its aspects the present invention provides a food article, wherein the food article comprises the lipid composition and/or the vegetable-derived fat source in accordance with the invention, as described herein above and below, for (or for use in or for use in a method for) facilitating catch up growth in a subject.

Yet in a further one of its aspects the present invention provides a food article, wherein the food article comprises the lipid composition and/or the vegetable-derived fat source in accordance with the invention, as described herein above and below, for (or for use in or for use in a method for) improvement of weight gain in a subject, e.g., during a catch up growth.

Yet in a further one of its aspects the present invention provides a food article, wherein the food article comprises the lipid composition and/or the vegetable-derived fat source in accordance with the invention, as described herein above and below, for (or for use in or for use in a method for) increasing of bone length in a subject, e.g., during a catch up growth.

Yet in a further one of its aspects the present invention provides a food article, wherein the food article comprises the lipid composition and/or the vegetable-derived fat source in accordance with the invention, as described herein above and below, for (or for use in or for use in a method for) increasing of growth plate (e.g., EGP) height in a subject, e.g., during a catch up growth.

Yet in a further one of its aspects the present invention provides a food article, wherein the food article comprises the lipid composition and/or the vegetable-derived fat source in accordance with the invention, as described herein above and below, for (or for use in or for use in a method for) increasing of growth plate (e.g., EGP) zone of proliferation in a subject, e.g., during a catch up growth.

Yet in a further one of its aspects the present invention provides a food article, wherein the food article comprises the lipid composition and/or the vegetable-derived fat source in accordance with the invention, as described herein above and below, for (or for use in or for use in a method for) increasing of growth plate (e.g., EGP) zone of maturation and hypertrophy in a subject, e.g., during a catch up growth.

Yet in a further one of its aspects the present invention provides a food article, wherein the food article comprises the lipid composition and/or the vegetable-derived fat source in accordance with the invention, as described herein above and below, for (or for use in or for use in a method for) increasing of Insulin like growth factor 1 levels in a subject, e.g., during a catch up growth.

Yet in a further one of its aspects the present invention provides a food article, wherein the food article comprises the lipid composition and/or the vegetable-derived fat source in accordance with the invention, as described herein above and below, for (or for use in or for use in a method for) reducing leptin levels in a subject, e.g., during a catch up growth.

Yet in a further one of its aspects the present invention provides a food article, wherein the food article comprises the lipid composition and/or the vegetable-derived fat source in accordance with the invention, as described herein above and below, for (or for use in or for use in a method for) improving metabolic processes in a subject, e.g., during a catch up growth.

Yet in a further one of its aspects the present invention provides a food article, wherein the food article comprises the lipid composition and/or the vegetable-derived fat source in accordance with the invention, as described herein above and below, for (or for use in or for use in a method for) reducing risk of obesity in a subject, e.g., during a catch up growth.

As used herein, the term “subject” refers to a healthy subject or a subject suffering from a specific disorder/condition or at risk of developing a specific disorder/condition. It is noted that the disorder and/or condition may be related or may be unrelated to a growth problem. Thus, at times, the subject may have a growth problem which may be a side effect associated with a specific disorder and/or condition.

In some embodiments according to the invention the subject may be a subject at risk of developing at least one growth problem. A subject at risk of developing a growth problem may be, but is not limited to, any one of: an undernourished subject (i.e., a subject not nourished with sufficient or proper food to maintain or promote health or normal growth); a subject with poor eating habits and nutrition; a subject with poor sleeping patterns; a subject suffering from an endocrine malfunction (hormones); a subject suffering from a chronic illness; a subject with intrauterine growth retardation (IUGR); a subject suffering from failure to thrive and/or inadequate weight gain anytime after birth; a subject suffering from genetic problem/s; a subject suffering from one or more of Down syndrome, organ problems, hormone problems and damage to the brain or central nervous system which may cause feeding difficulties in said subject e.g., an infant; a subject suffering from heart or lung problems which may affect how nutrients move through the body; a subject suffering from anemia or other blood disorders; a subject suffering from one or more of chronic infections, metabolic disorders, problems during embrionic phase or low birth weight; a subject suffering from one or more of poor environment conditions e.g., emotional issues, poverty, malnutrition, poor nutrition, exposure to infections, parasites, or toxins; a subject suffering from poor eating habits; a subject consuming drugs that may affect eating habits; a subject suffering from one or more of chronic diseases such as diabetes, congenital heart disorders, sickle cell disease, chronic kidney failure, cystic fibrosis, crohn, inflammattory bowel disease, celiac and rheumatoid arthritis; a subject suffering from one or more of abnormal formation and/or growth of cartilage and/or bone, skeletal dysplasia and chondrodystrophies.

In some embodiments according to the invention the subject may or may not suffer from gastrointestinal problems that may interfere with nutrients absorption.

In some embodiments according to the invention the subject may be any one of an infant (preterm or term, newborn from the day of birth, to age of about 12 months i.e., about 1 year), a toddler (from about one year up to about the age of 3), a child (from about 3 years to about 12 years and an adolescent (from 12 years to about 18 years).

In some embodiments of the invention the subject is an infant.

In some embodiments of the invention the subject is a toddler.

In some embodiments of the invention the subject is a child.

In some embodiments of the invention the subject is an adolescent.

In some embodiments according to the invention the infant may be any one of pre-term infant or term infant; a small for gestation age (SGA) infant; an appropriate for gestation age (AGA) infant large for gestation age (LGA) infant; an infant born by regular (vaginal) delivery, cesarean surgery (Caesarean section) or any other modes of delivery.

As used herein the term “newborn” includes pre-mature infants, post-mature infants and full term newborns.

In some embodiments of the invention the infant may be an infant at very early infancy e.g., newborn, infant at age of up to 6 weeks or up to 12 weeks or at the age of between about 6 to 12 weeks. In some embodiments of the invention the infant may be at the age of 12 weeks or above, at times at the age of between about 12 to 24 weeks and even at times at the age of 24 weeks or above. Further, at times the infant may be at the age of between about 24 weeks to about 12 months.

In some embodiments of according to the invention the subject is an infant, a child or an adolescent.

In some further embodiments according to the invention the subject is a child or an infant.

In the various aspects and embodiments of the invention, the lipid composition and/or the vegetable-derived fat source according to the invention may be provided to the infant for a period of time from day one to weeks, months, years, etc. following birth.

In some embodiments of the invention the subject may be a healthy subject experiencing growth problem/s such as for example insufficient growth (e.g., reduced growth rate). The subject may also be a non-healthy subject experiencing growth problem/s such as for example insufficient growth (e.g., reduced growth rate).

In some embodiments according to the invention the subject may experience one or more growth problem/s.

In a specific embodiment of the invention the lipid composition and/or the vegetable-derived fat source of the invention is for (or for use, or for use in a method for) improving growth in a subject, particularly an infant, more particularly an infant at very early infancy e.g., newborn, infant at age of up to 6 weeks or up to 12 weeks or up to 24 weeks or up to 12 months. In a specific embodiment the subject is a toddler. In a specific embodiment the subject is a child. In a specific embodiment the subject is an adolescent.

In a specific embodiment the compositions, methods or uses disclosed herein are for improving growth in a subject, particularly a subject in catch up period. To this end, the compositions, methods or uses disclosed herein are for facilitating catch up growth in the subject.

Thus, in some embodiments according to the invention the subject is a subject in catch up period.

In a specific embodiment according to the invention the subject in catch up period is an infant, more particularly an infant at very early infancy e.g., newborn, infant at age of up to 6 weeks or up to 12 weeks or up to 24 weeks or up to 12 months.

In a specific embodiment the subject in catch up period is a toddler.

In a specific embodiment the subject in catch up period is a child.

In a specific embodiment the subject in catch up period is an adolescent.

In another one of its aspects the present invention provides a food article, wherein the food article comprises the lipid composition and/or the vegetable-derived fat source in accordance with the invention, as described herein above and below, for (or for use in or for use in a method for) improving growth in a subject, particularly an infant, more particularly an infant at very early infancy e.g., newborn, infant at age of up to 6 weeks or up to 12 weeks or up to 24 weeks. In a specific embodiment the subject is a toddler. In a specific embodiment the subject is a child. In a specific embodiment the subject is an adolescent.

In another one of its aspects the present invention provides a food article, wherein the food article comprises the lipid composition and/or the vegetable-derived fat source in accordance with the invention, as described herein above and below, for (or for use in or for use in a method for) improving growth in a subject in catch up growth, particularly an infant, more particularly an infant at very early infancy e.g., newborn, infant at age of up to 6 weeks or up to 12 weeks or up to 24 weeks. In a specific embodiment the subject in catch up period is a toddler. In a specific embodiment the subject in catch up period is a child. In a specific embodiment the subject in catch up period is an adolescent.

In some embodiments the compositions, fat source, methods or uses disclosed herein are for improving growth in an infant, wherein improving growth being one or both of increasing growth rate and increasing final height.

In a specific embodiment of the invention the edible lipid composition of the invention and/or the vegetable-derived fat source of the invention is for (or for use, or for use in a method for) treating at least one growth problem in a subject, particularly an infant, more particularly an infant at the age of up to 12 weeks, more particularly an infant at the age of 12 weeks or above, even more particularly at the age of between about 12 to 24 weeks and even more particularly at the age of 24 weeks or above. In a specific embodiment the subject is a toddler. In a specific embodiment the subject is a child. In a specific embodiment the subject is an adolescent.

In a specific embodiment of the invention the edible lipid composition of the invention and/or the vegetable-derived fat source of the invention is for (or for use, or for use in a method for) treating at least one growth problem in a subject in catch up period, particularly an infant, more particularly an infant at the age of up to 12 weeks, more particularly an infant at the age of 12 weeks or above, even more particularly at the age of between about 12 to 24 weeks and even more particularly at the age of 24 weeks or above. In a specific embodiment the subject in catch up period is a toddler. In a specific embodiment the subject in catch up period is a child. In a specific embodiment the subject in catch up period is an adolescent.

In a specific embodiment the method of the invention is for treating at least one growth problem in a subject, particularly an infant, more particularly an infant at the age of up to 12 weeks, more particularly an infant at the age of 12 weeks or above, even more particularly at the age of between about 12 to 24 weeks and even more particularly at the age of 24 weeks or above. In a specific embodiment the subject is a toddler. In a specific embodiment the subject is a child. In a specific embodiment the subject is an adolescent.

In a specific embodiment the method of the invention is for treating at least one growth problem in a subject in catch up period, particularly an infant, more particularly an infant at the age of up to 12 weeks, more particularly an infant at the age of 12 weeks or above, even more particularly at the age of between about 12 to 24 weeks and even more particularly at the age of 24 weeks or above. In a specific embodiment the subject in catch up period is a toddler. In a specific embodiment the subject in catch up period is a child. In a specific embodiment the subject in catch up period is an adolescent.

In another one of its aspects the present invention provides a food article, wherein the food article comprises the lipid composition and/or the vegetable-derived fat source in accordance with the invention, as described herein above and below, for (or for use in or for use in a method for) treating at least one growth problem in a subject, particularly an infant, more particularly an infant at the age of up to 12 weeks, more particularly an infant at the age of 12 weeks or above, even more particularly at the age of between about 12 to 24 weeks and even more particularly at the age of 24 weeks or above. In a specific embodiment the subject is a toddler. In a specific embodiment the subject is a child. In a specific embodiment the subject is an adolescent.

In another one of its aspects the present invention provides a food article, wherein the food article comprises the lipid composition and/or the vegetable-derived fat source in accordance with the invention, as described herein above and below, for (or for use in or for use in a method for) treating at least one growth problem in a subject in catch up period, particularly an infant, more particularly an infant at the age of up to 12 weeks, more particularly an infant at the age of 12 weeks or above, even more particularly at the age of between about 12 to 24 weeks and even more particularly at the age of 24 weeks or above. In a specific embodiment the subject in catch up period is a toddler. In a specific embodiment the subject in catch up period is a child. In a specific embodiment the subject in catch up period is an adolescent.

In some embodiments the compositions, fat sources methods or uses disclosed herein are for treating at least one growth problem in an infant, wherein the growth problem is insufficient growth rate compared for example to acceptable norms at the specific subject's gender and/or age and/or health condition and the like and/or at times weight.

In some embodiments according to the invention the subject may be a healthy subject (e.g., an infant and/or a child) suffering from at least one growth problem such as insufficient growth or insufficient growth rate compared for example to acceptable norms at the specific subject's gender and/or age and/or health condition and the like and/or optionally weight.

In some embodiments according to the invention the subject may be a non-healthy subject (e.g., an infant and/or a child) suffering from at least one growth problem such as insufficient growth or insufficient growth rate compared for example to acceptable norms at the specific subject's gender and/or age and/or health condition and the like and/or optionally weight.

In the context of the present invention the term “treatment” or “treating” and the like are used herein to refer to obtaining a desired pharmacological or physiological effect on the subject, including prophylactic in terms of “preventing” or partially preventing an undesired condition or symptoms from developing and/or therapeutic in terms of “curing” partial or complete curing of an already existing undesired condition or in a subject that is in risk for developing an undesired condition that may affect growth. The term “treating” is used within the context of the present disclosure as treatment of subjects who are healthy and/or suffer from a disorder, disease, or impaired physiological/medical growth related condition.

In the various aspects and embodiments of the invention, the triglycerides according to the invention may comprise saturated and/or mono-unsaturated and/or polyunsaturated fatty acids residues.

In the various aspects and embodiments of the invention, the fatty acid residues at the sn-2 position of the glycerol backbone may be a saturated fatty acid residue, including C₈ to C₂₄, and in some particular embodiments C₁₄-C₁₈ fatty acid residues.

In the various aspects and embodiments of the invention, the saturated fatty acid may be any one of butyric acid (butanoic acid, C4:0), caproic acid (hexanoic acid, C6:0), caprylic acid (octanoic acid, C8:0), capric acid (decanoic acid, C10:0), lauric acid (dodecanoic acid, C12:0), myristic acid (tetradecanoic acid, C14:0), palmitic acid (hexadecanoic acid, C16:0), stearic acid (octadecanoic acid, C18:0), arachidic acid (eicosanoic acid, C20:0) and behenic acid (docosanoic acid C22:0).

In some specific embodiments, according to all aspects of the invention, the saturated fatty acid residue is predominantly a palmitic acid residue.

In the various aspects and embodiments of the invention, in the vegetable-derived fat source according to the invention at least about 30%, at time at least about 33%, at times at least about 38%, at times at least about 40%, at times at least about 43%, at times at least about 50% of the total palmitic acid residues are present at the sn-2 position of the glycerol backbone. In some further embodiments of the invention the level of palmitic acid moieties at the sn-2 position of the glycerol backbone is 30%-70% of total palmitic acid. In yet some further embodiments of the invention the level of palmitic acid moieties at the sn-2 position of the glycerol backbone is 40%-70% of total palmitic acid. In yet some further embodiments of the invention the level of palmitic acid moieties at the sn-2 position of the glycerol backbone is 40%-60% of total palmitic acid. In some further embodiments of the invention the level of palmitic acid moieties at the sn-2 position of the glycerol backbone is 43%-60% of total palmitic acid. In yet some further embodiments of the invention the level of palmitic acid moieties at the sn-2 position of the glycerol backbone is 50%-65% of total palmitic acid. In yet some further embodiments of the invention the level of palmitic acid moieties at the sn-2 position of the glycerol backbone is 40%-55% of total palmitic acid.

In the various aspects and embodiments of the invention, in the vegetable-derived fat source according to the invention at least about 20%, at times at least about 23%, at times at least about 30%, at times at least about 40%, at times 40%-80%, at times 50%-55% of the total fatty acid moieties at the sn-2 position of the glycerol backbone are saturated.

In some embodiments according to the present invention, in the vegetable-derived fat source according to the invention at least about 20% of the total fatty acid moieties at the sn-2 position of the glycerol backbone are saturated.

In the various aspects and embodiments of the invention, in the vegetable-derived fat source according to the invention at least about 10%, at times at least about 15%, at times at least about 20%, at times at least about 22%, at times 15%-65%, at times 20%-40%, at times 20%-35%, at times 20%-30%, of the total fatty acid moieties at the sn-2 position of the glycerol backbone are palmitic acid.

In the various aspects and embodiments of the invention, in the vegetable-derived fat source according to the invention at most about 40%, at times at most about 36%, at times at most about 20%, at times 5%-40%, at times 9%-36% of the total fatty acid moieties at the sn-1 and sn-3 positions of the glycerol backbone are palmitic acid.

In the various aspects and embodiments of the invention, in the vegetable-derived fat source according to the invention at most about 70%, at times at most about 60%, at times at most about 47%, at times 30%-70%, at times 40%-65%, at times 45%-65%, at times 45%-61%, of the total palmitic acid are esterified at the sn-1 and sn-3 positions of the glycerol backbone.

In the various aspects and embodiments of the invention, in the vegetable-derived fat source according to the invention at most about 35%, at times 10%-35%, at times 25%-35% of the total fatty acid moieties at the sn-2 position of the glycerol backbone are oleic acid.

In the various aspects and embodiments of the invention, in the vegetable-derived fat source according to the invention at most about 35%, at times at most about 27.2%, at times 15%-35%, at times 20%-30% of the total oleic acid moieties are at the sn-2 position of the glycerol backbone.

In the various aspects and embodiments of the invention, in the vegetable-derived fat source according to the invention at most about 80%, at times at most about 76%, at times 20%-76%, at times 25%-76%, at times 25-50% of the total fatty acid moieties at the sn-2 position of the glycerol backbone are unsaturated fatty acids.

In the various aspects and embodiments of the invention, in the vegetable-derived fat source according to the invention at most about 50%, at times at most about 45%, at times at most about 40%, at times at most about 25%, at times at most about 15%, at times 10%-50%, at times 10%-45%, at times 14%-40% of the total fatty acid moieties, at the sn-1 and sn-3 positions of the glycerol backbone are saturated.

In the various aspects and embodiments of the invention, in the vegetable-derived fat source according to the invention at least about 50%, at times at least about 60%, at times at least about 70%, at times 50%-80%, at times 60%-75% of the total fatty acid moieties at the sn-1 and sn-3 positions of the glycerol backbone are unsaturated.

In some embodiments according to the present invention, in the vegetable-derived fat source according to the invention at least about 50% of the total fatty acid moieties at the sn-1 and sn-3 positions of the glycerol backbone are unsaturated.

In the various aspects and embodiments of the invention, in the vegetable-derived fat source according to the invention at most about 1%, at times at most about 0.5%, at times at most about 0.2%, at times at most 0.1% of the total fatty acid moieties are free fatty acids.

In the various aspects and embodiments of the invention, in the vegetable-derived fat source according to the invention at most about 5%, at times at most about 4%, at times at most about 3%, at times at most 2% of the total fatty acid moieties are in form of diacylglycerides.

In the various aspects and embodiments of the invention, in the vegetable-derived fat source according to the invention at most about 3%, at times at most about 1%, at times at most about 0.5%, at times at most 0.1% of the total fatty acid moieties are in form of monoacylglycerides.

In the various aspects and embodiments of the invention an unsaturated fatty acid may be any one of oleic acid (C18:1), linoleic acid (C18:2), α-linolenic acid (C18:3) and gadoleic acid (C20:1).

In the various aspects and embodiments of the invention, in the vegetable-derived fat source according to the invention at least about 40%, at times at least about 50%, at times at least about 60%, at times 40-80%, at times 50%-60% of the total fatty acids are unsaturated fatty acid moieties.

In some embodiments according to the present invention, in the vegetable-derived fat source according to the invention at least about 40% of the total fatty acids are unsaturated fatty acid moieties.

In the various aspects and embodiments of the invention, in the vegetable-derived fat source according to the invention at least about 50%, at times at least 65% of the unsaturated fatty acids are oleic acid, at times 50%-90%, at times 80%-90% of the unsaturated fatty acids are oleic acid.

In some embodiments according to the present invention, in the vegetable-derived fat source according to the invention at least about 50% of the unsaturated fatty acids are oleic acid.

In some embodiments of the invention, in the vegetable-derived fat source according to the invention at least about 5%, at times at least 10%, at times at least about 15%, at times at least 20%, at times at least about 46%, at times 10%-60%, at times 10%-50%, at times 15%-55%, at times 15%-35%, at times 10%-15%, at times 40%-50% of the unsaturated fatty acids are linoleic acid.

In some embodiments according to the present invention, in the vegetable-derived fat source according to the invention at least about 15% of the unsaturated fatty acids are linoleic acid.

In some further embodiments of the invention, in the vegetable-derived fat source according to the invention at most about 5%, at times at most 2.5%, at times at most 2%, at times 0.1%-8%, at times 0.1%-4%, at times 0.1%-3%, at times 0.2%-2.5% and at times 0.4%-2% of the unsaturated fatty acids are linolenic acid.

In some embodiments according to the present invention, in the vegetable-derived fat source according to the invention at most about 5% of the unsaturated fatty acids are linolenic acid.

In the various aspects and embodiments of the invention, in the vegetable-derived fat source according to the invention at least about 35%, at times at least about 40%, at times at least about 57% of the unsaturated fatty acid moieties at the sn-1 and sn-3 positions are oleic acid moieties. In some further embodiments of the invention, in the vegetable-derived fat source according to the invention 40%-90%, at times 50%-90%, at times 50%-70%, at times 70%-90% of the unsaturated fatty acid moieties at the sn-1 and sn-3 positions are oleic acid moieties.

In some embodiments according to the present invention, in the vegetable-derived fat source according to the invention at least about 35% of the unsaturated fatty acid moieties at the sn-1 and sn-3 positions are oleic acid moieties.

In the various aspects and embodiments of the invention, in the vegetable-derived fat source according to the invention at least about 4%, at times at least about 6%, at times 6%-50%, at times 10%-45%, at times 10%-40%, at times 10%-30%, at times 10%-35% of the unsaturated fatty acid moieties at the sn-1 and sn-3 positions are linoleic acid moieties.

In some embodiments according to the present invention, in the vegetable-derived fat source according to the invention at least about 4% of the unsaturated fatty acid moieties at the sn-1 and sn-3 positions are linoleic acid moieties.

In the various aspects and embodiments of the invention, the vegetable-derived fat source according to the invention is characterized by having the following parameters: (i) at least 20%, at times at least 30%, at times at least 33%, at times at least 38%, and even at times at least 40% of the total palmitic acid residues are at the sn-2 position of the glycerol backbone; (ii) at least 50%, at times at least 60%, at least 70% of the fatty acid moieties at the sn-1 and sn-3 positions of the glycerol backbone are unsaturated; (iii) at least 35%, at times at least 40%, at least about 50% of the unsaturated fatty acid moieties at the sn-1 and sn-3 positions are oleic acid moieties; and (iv) at least 4%, at times at least 6%, of the unsaturated fatty acid moieties at the sn-1 and sn-3 positions are linoleic acid moieties.

In the various aspects and embodiments of the invention, the vegetable-derived fat source according to the invention comprises triglycerides with at most 50%, at times at most 38%, at times about 10%-50%, at times 15%-50%, at times 15%-40%, at times 15%-38%, at times 15%-33%, at times 15%-25%, at times 17%-24%, at times 17%-23%, at times 18%-23%, at times 18%-22%, at times 19%-22% and at times 19%-21%, at times 13%-50%, at times 13%-40%, at times 13%-38%, at times 13%-33%, at times 13%-25%, at times 13%-20%, at times 13%-18% palmitic acid moieties out of the total fatty acids. In some embodiments of the invention the vegetable-derived fat source comprises triglycerides with about 20%-50%, at times 25%-40%, at times 30%-40%, at times 30%-38%, at times 30%-35%, at times 31%-34% and at times 31%-33% palmitic acid moieties out of the total fatty acids.

Thus, the palmitic acid content of the fat source according to the invention may be 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54% or 55% of the total fatty acids.

In the various aspects and embodiments of the invention, in the vegetable-derived fat source according to the invention at least about 13% w/w, at times at least about 15%, at times at least about 18%, at times at least about 22%, at times 18%-60%, at times 22%-50%, at times 20%-35% of the total fatty acid residues at the sn-2 position of the glycerol backbone are palmitic acid residues. In some embodiments of the invention, in the vegetable-derived fat source according to the invention at least 50%, at times at least 60%, at times 50-80%, at times 60-90%, at times 60%-70% of the fatty acid moieties at the sn-2 position of the glycerol backbone are palmitic acid residues.

In the various aspects and embodiments of the invention, in the vegetable-derived fat source the level of palmitic acid moieties at the sn-2 position of the glycerol backbone is 40%-60% of total palmitic acid, 20%-30% of the total fatty acid moieties at the sn-2 position of the glycerol backbone are palmitic acid, 45%-61% of the total palmitic acid are esterified at the sn-1 and sn-3 positions of the glycerol backbone, 25%-35% of the total fatty acid moieties at the sn-2 position of the glycerol backbone are oleic acid, 20%-30% of the total oleic acid moieties are at the sn-2 position of the glycerol backbone, 10%-45% of the total fatty acid moieties, at the sn-1 and sn-3 positions of the glycerol backbone are saturated and 50%-80% of the total fatty acid moieties at the sn-1 and sn-3 positions of the glycerol backbone are unsaturated.

A non-limiting example of a lipid composition or the vegetable-derived fat source according to all aspects of the invention comprises:

0%-10%—C8:0 fatty acid residue out of the total fatty acid residue content;
0%-10%—C10:0 fatty acid residue out of the total fatty acid residue content;
0%-22%—C12:0 fatty acid residue out of the total fatty acid residue content;
0%-15%—C14:0 fatty acid residue out of the total fatty acid residue content;
13%-55%—C16:0 fatty acid residue out of the total fatty acid residue content;
1%-13%—C18:0 fatty acid residue out of the total fatty acid residue content;
20%-75%—C18:1 fatty acid residue out of the total fatty acid residue content;
2%-40%—C18:2 fatty acid residue out of the total fatty acid residue content; and
0%-8%—C18:3 fatty acid residue out of the total fatty acid residue content, and
wherein at least 20%, at times at least 30%, at times at least 33%, and even at times at least 40%, at times 40%-70%, even at times 40%-60% or 43%-60% of the C16:0 fatty acid residue out of the total fatty acid residue content is at sn-2 position the glycerol backbone.

A further non-limiting example of a lipid composition or the vegetable-derived fat source according to all aspects of the invention comprises:

0%-10%—C8:0 fatty acid residue out of the total fatty acid residue content;
0%-10%—C10:0 fatty acid residue out of the total fatty acid residue content;
0%-22%—C12:0 fatty acid residue out of the total fatty acid residue content;
0%-15%—C14:0 fatty acid residue out of the total fatty acid residue content;
15%-55%—C16:0 fatty acid residue out of the total fatty acid residue content;
1%-7%—C18:0 fatty acid residue out of the total fatty acid residue content;
20%-75%—C18:1 fatty acid residue out of the total fatty acid residue content;
2%-40%—C18:2 fatty acid residue out of the total fatty acid residue content; and
0%-8%—C18:3 fatty acid residue out of the total fatty acid residue content, and
wherein at least 20%, at times at least 30%, at times at least 33%, and even at times at least 40%, at times 40%-70%, even at times 40%-60% or 43%-60% of the C16:0 fatty acid residue out of the total fatty acid residue content is at sn-2 position the glycerol backbone.

In accordance with a more particular embodiment, the lipid composition or the vegetable-derived fat source according to all aspects of the invention comprises:

0%-2%—C8:0 fatty acid residue out of the total fatty acid residue content;
0%-2%—C10:0 fatty acid residue out of the total fatty acid residue content;
5%-15%—C12:0 fatty acid residue out of the total fatty acid residue content;
2%-10%—C14:0 fatty acid residue out of the total fatty acid residue content;
17%-25%—C16:0 fatty acid residue out of the total fatty acid residue content;
2%-5%—C18:0 fatty acid residue out of the total fatty acid residue content;
28%-48%—C18:1 fatty acid residue out of the total fatty acid residue content;
5%-20%—C18:2 fatty acid residue out of the total fatty acid residue content;
1%-3%—C18:3 fatty acid residue out of the total fatty acid residue content; and
wherein at least 20%, at times at least 30%, at times at least 33%, and even at times at least 40%, at times 40%-70%, even at times 40%-60% or 43%-60% of the C16:0 fatty acid residue out of the total fatty acid residue content is at sn-2 position the glycerol backbone.

Thus, the vegetable-derived fat source according to the invention may comprise: 0%, 1%, 2%, 3%, 4%, 5%, 6%, 7%, 8%, 9% or 10% of C8:0 fatty acids of the total fatty acids; 0%, 1%, 2%, 3%, 4%, 5%, 6%, 7%, 8%, 9% or 10% of C12:0 fatty acids of the total fatty acids; 0%, 1%, 2%, 3%, 4%, 5%, 6%, 7%, 8%, 9%, 10%, 11%, 12%, 13%, 14%, 15%, 16%, 17%, 18%, 19%, 20%, 21% or 22% of C12:0 fatty acids of the total fatty acids; 0%, 1%, 2%, 3%, 4%, 5%, 6%, 7%, 8%, 9%, 10%, 11%, 12%, 13%, 14% or 15% C14:0 fatty acids of the total fatty acids; 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54% or 55% C16:0 fatty acids of the total fatty acids; 1%, 1.2%, 1.4%, 1.6%, 1.8%, 2%, 2.2%, 2.4%, 2.6%, 2.8%, 3%, 3.2%, 3.4%, 3.6%, 3.8%, 4%, 4.2%, 4.4%, 4.6%, 4.8%, 5%, 5.2%, 5.4%, 5.6%, 5.8%, 6%, 6.2%, 6.4%, 6.6%, 6.8%, 7%, 8%, 9%, 10%, 11%, 12% or 13% C18:0 fatty acids of the total fatty acids; 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74% or 75% C18:1 fatty acids of the total fatty acids; 2%, 3%, 4%, 5%, 6%, 7%, 8%, 9%, 10%, 11%, 12%, 13%, 14%, 15%, 16%, 17%, 18%, 19%, 20%, 22%, 23%, 25%, 26%, 27%, 28%, 29%, 30%, 31%, 32%, 33%, 34%, 35%, 36%, 37%, 38%, 39%, 40% C18:2 fatty acids of the total fatty acids; 0%, 0.5%, 1%, 1.2%, 1.4%, 1.6%, 1.8%, 2%, 2.2%, 2.4%, 2.6%, 2.8%, 3%, 3.2%, 3.4%, 3.6%, 3.8%, 4%, 4.2%, 4.4%, 4.6%, 4.8%, 5%, 5.2%, 5.4%, 5.6%, 5.8%, 6%, 6.2%, 6.4%, 6.6%, 6.8%, 7%, 7.2%, 7.5%, 7.8% or 8% C18:3 fatty acids of the total fatty acids.

Of particular interest are vegetable-derived fat sources which are based on edible synthetic oils (which can be enzymatically produced), which mimic, as are (designated herein as fat base), or when blended with edible vegetable oil/s (designated herein as fat blend), the triglyceride composition of human breast milk fat. Such fat sources have a high level of palmitic acid at the sn-2 position of the triglycerides, and a high level of unsaturated fatty acids at sn-1 and sn-3 positions.

Said edible vegetable oil/s which may be blended with said fat base to prepare the fat blend may be: natural vegetable oil/s, randomized vegetable oil/s, interesterified vegetable oils, enzymatically interesterified vegetable oils, at least two vegetable oils which were co-randomized, at least two vegetable oils which were co-interesterified. Said edible vegetable oil/s comprises oils selected from the group consisting of but not limited to soybean oil, high oleic sunflower oil, high oleic safflower oil palm oil, palm olein oil, canola oil, coconut oil, palm kernel oil, sunflower oil, corn oil, safflower and rapeseed oil.

The lipid composition and/or vegetable-derived fat source of the invention may be used in the preparation of pharmaceutical, nutritional or nutraceutical composition, or a functional or medical food.

The lipid composition and/or vegetable-derived fat source of the invention may be used in the preparation of infant formula, toddler formula, child formula, adolescent formula, dietary supplements and food articles.

In some embodiments the lipid composition and/or vegetable-derived fat source of the invention may be used in the preparation of infant formula.

In some embodiments the lipid composition and/or vegetable-derived fat source of the invention may be used in the preparation of toddler formula.

In some embodiments the lipid composition and/or vegetable-derived fat source of the invention may be used in the preparation of child formula.

In some embodiments the lipid composition and/or vegetable-derived fat source of the invention may be used in the preparation of adolescent formula.

Thus, in the various aspects and embodiments of the invention, the vegetable-derived fat source may be man-made, synthetically made, artificially made and/or enzymatically made.

Further, in the various aspects and embodiments of the invention, the vegetable-derived fat source and/or at least one triglyceride of the fat source may be selected from the group consisting of naturally occurring triglycerides, synthetic triglycerides, semi-synthetic triglycerides, and artificially produced triglycerides. In some further embodiments the triglyceride may be obtained from a vegetable source.

The vegetable-derived fat source according to the invention can also be a substitute human milk fat composition or human milk fat mimetic composition comprising a blend of at least 25%, at times 25-83%, at times 30-70% of a fat base concentrate with up to 75%, at times 17-75%, at times 20-75%, at times 30-70% of at least one edible vegetable oil. In some specific embodiments the fat source may comprise 25%, 30%, 36%, 50%, 52%, 60%, 63%, 73% and 83% of the fat base concentrate and 75%, 70%, 64%, 50%, 48%, 40%, 37%, 27% and 17%, respectively, of the at least one vegetable oil.

Most importantly, the lipid composition and/or vegetable-derived fat source of the present invention may be used in the preparation of infant formula, toddler formula or child formula.

Thus, in a further one of its aspects the present invention provides a formula (e.g., an infant formula) comprising the composition and/or vegetable-derived fat source according to the invention for (or for use in or for use in a method for) one or both of improving growth and treating at least one growth problem in a subject (e.g., infant, toddler or child) administered with said formula.

In a further one of its aspects the present invention provides a formula (e.g., an infant formula, a child formula) comprising the composition and/or vegetable-derived fat source according to the invention for (or for use in or for use in a method for) facilitating catch up growth in a subject (e.g., an infant, a child) administered with said formula.

In yet a further one of its aspects the present invention provides a formula (e.g., an infant formula) comprising the composition and/or vegetable-derived fat source according to the invention for (or for use in or for use in a method for) improving growth in a subject (e.g., an infant) administered with said formula.

In yet a further one of its aspects the present invention provides a formula (e.g., an infant formula) comprising the composition and/or vegetable-derived fat source according to the invention for (or for use in or for use in a method for) treating at least one growth problem in a subject (e.g., an infant) administered with said formula.

In yet a further one of its aspects the present invention provides an infant formula comprising the composition and/or vegetable-derived fat source according to the invention for (or for use in or for use in a method for) improving growth in said infant.

Yet in a further one of its aspects the present invention provides an infant formula comprising the composition and/or vegetable-derived fat source according to the invention for (or for use in or for use in a method for) treating at least one growth problem in said infant.

Yet in a further one of its aspects the present invention provides an infant formula comprising the composition and/or vegetable-derived fat source according to the invention for (or for use in or for use in a method for) for facilitating catch up growth in said infant.

The formula (e.g., infant formula) used by the present invention may comprise in addition to the fat source at least one protein component and optionally at least one of carbohydrate source, vitamins, minerals, nucleotides and amino acids.

Thus, in the various aspects and embodiments of the invention, the formula (e.g., infant formula) comprises the lipid composition and/or vegetable-derived fat source, together with a protein source, a carbohydrate source, minerals, vitamins and optionally at least one of carrier, diluent, additive or excipient.

The lipid composition and/or vegetable-derived fat source of the present invention may be used in the preparation of a food article. The food article used by the invention comprises in addition to the fat source at least one protein component and optionally at least one of carbohydrate source, vitamins, minerals, and amino acids.

Thus, in the various aspects and embodiments of the invention, the food article comprises the lipid composition and/or vegetable-derived fat source, together with a protein source, a carbohydrate source, minerals, vitamins and optionally at least one of carrier, diluent, additive or excipient.

The lipid composition and/or vegetable-derived fat source of the present invention may be used in the preparation of a child formula. The child formula used by the invention comprises in addition to the fat source at least one protein component and optionally at least one of carbohydrate source, vitamins, minerals, and amino acids.

Thus, in the various aspects and embodiments of the invention, the child formula comprises the lipid composition and/or vegetable-derived fat source, together with a protein source, a carbohydrate source, minerals, vitamins and optionally at least one of carrier, diluent, additive or excipient.

The terms “lipid” and “fat” are used herein synonymously.

The methods according to the invention are best practiced through administering to a subject, a formula, an infant formula, toddler formula, child formula, clinical nutrition product or a food article prepared with and comprising the lipid composition and/or vegetable-derived fat source as described in the invention, either in the form of a concentrate base or in the form of a blend. Non-limiting examples of a fat concentrate/base are Fat Bases 1 to 11 in Table 1 of Example 1, and non-limiting examples of Fat blends are described in Table 2-6 of Example 1.

The lipid composition and/or vegetable-derived fat source according to the invention may be used in the preparation of or as part of a nutritional, pharmaceutical, or nutraceutical composition or a functional or medical food.

A nutritional composition as used herein can be any nutritional composition including, but not limited to: human milk fat substitute, formula, infant formula, toddler formula, child formula, dairy product, including milk and dairy drinks, milk powder, drinks, shakes, ice cream, biscuit, soy product, bakery, pastry, bread, cake, sauce, soup, prepared food, including prepared mashed vegetables and/or fruits, frozen food, condiment, confectionary, oil, fat, margarine, spread, filling, meat product, cereal, instant product, instant drink product, infant food, toddler food, bar, snack, candy, and chocolate product.

A functional food as used herein can be any functional food, including, but not limited to: dairy product, ice-cream, biscuit, soy product, bakery, pastry, cakes and bread, instant product, sauce, soup, prepared food, frozen food, condiment, confectionary, oils and fat, margarine, spread, filling, cereal, instant product, drinks and shake, infant food, bar, snack, candy, and chocolate product.

A nutraceutical composition as used herein can be any nutraceutical, which can be any substance that may be considered as a food or part of a food and provides medical or health benefits, including the prevention and treatment of diseases or disorders. Such nutraceutical compositions include, but are not limited to: a food additive, a food supplement, a dietary supplement, genetically engineered foods (such as for example vegetables, herbal products, and processed foods such as cereals, soups, and beverages), stimulant functional food, clinical nutrition product, medical food, and pharmafood. Dietary supplements may be delivered in the form of soft gel capsules, tablets, syrups, and other known dietary supplement delivery systems.

The pharmaceutical or nutraceutical compositions may be in any of the many dosage delivery forms commonly used in the art. Pharmaceutical compositions suitable for oral administration may be presented as discrete dosage units (such as pills, tablets, pellets, dragèes, capsules, or softgel), as a powder or granule, or as a solution, suspension, syrup, or elixir.

A medical food as used herein is specially formulated and intended for the dietary management of a disease/disorder that has distinctive nutritional needs that cannot be met by normal diet alone.

According to a specific embodiment of the invention, provided are infant formulas, toddler formulas and/or child formulas containing any one of the oil blends of the invention.

Administration is usually via oral or enteral route, which may include the use of gavage feeding, with a gastric feeding tube, sonda, etc, particularly where adapted for preterm infant feeding, infant feeding or for clinical nutrition.

The lipid composition and/or vegetable-derived fat source according to the invention may be formulated as or into an edible product. To this end, the lipid/fat source composition may be combined with at least one probiotic and prebiotic substance.

The edible product may be provided in fluid form (e.g. as a drink or beverage), as well as in a solid (e.g. as a powder) or semi solid form (e.g. as a porridge, or solid edible product). In a further aspect, the invention relates to a commercial package for preparing an edible fat source or food article which is recommended for improving growth and/or treating at least one growth problem in a subject, in accordance with the invention. In addition to the active and non-active constituents, the commercial package contains instructions for use. These include terms of storage, instructions for preparation of the fat source or food article for administration, required dilutions, dosages, frequency of administration and the like. A commercial package in accordance with the invention may also contain the lipid composition and/or vegetable-derived fat source in a ready-to-use form, together with instructions for use. Dosages are usually determined according to age, weight, sex and condition of the subject, in accordance to good medical practice known to the attending physician and other medical personnel.

Thus, in yet a further one of its aspects, the present invention provides a commercial package comprising:

a) an edible lipid composition and/or vegetable-derived fat source as herein disclosed which upon enteral administration to a subject improves growth in a subject and/or treats at least one growth problem in a subject;
b) optionally, at least one of edible physiologically acceptable protein, carbohydrate, vitamin, mineral and active or non-active additives;
c) optionally, at least one edible physiologically acceptable carrier or diluent for carrying the constituent/s defined in a) and b);
d) means and receptacles for admixing the constituents defined in a), b) and/or c); and
e) instructions for use.

In some embodiments the lipid composition of the invention may be artificially enriched with at least one triglyceride. As used herein, the term “artificially enriched” is used to denote that the lipid composition, while typically originated from a natural lipid source, is subjected to at least one modification, typically an enzymatic processing step, albeit not limited thereto, that promotes enrichment of the lipids with at least one triglyceride as defined.

The natural lipid source may be any edible lipid source, preferably, a vegetable oil, including, without being limited thereto, soybean oil, high oleic sunflower oil, high oleic safflower oil palm oil, palm olein oil, canola oil, coconut oil, palm kernel oil, sunflower oil, corn oil, safflower and rapeseed oil.

The lipid composition and/or vegetable-derived fat source of the invention is preferably provided to the subject orally, e.g. as an edible product, as discussed herein.

The methods according to the invention may be short-term methods as well as long-term methods. In other words, the subject, in particular, the infant, toddler or child subject, may receive a single dose of the lipid composition (or fat source) or an edible product comprising the same, as well as a series of doses of the lipid composition (or fat source), per day, a series of doses along a period of several days, weeks, months and 1, 2, 3 or more years. It is appreciated that when the methods according to the invention are conducted for a long period of time, the composition and/or the fat source and/or the product may vary depending on the age of the subject, as well as other considerations such as nutritional needs. Administration may commence at any time from day one after birth. Administration may also be to a breastfed subject, as supplementary feedings, or during or after weaning, or when the breastfeeding person (usually mother) is absent or unable to breastfeed.

In some embodiments the triglyceride according to the invention is selected from the group consisting of naturally occurring triglycerides, synthetic triglycerides semi-synthetic triglycerides, and artificially produced triglycerides, all derived from a vegetable source.

As used herein, the forms “a”, “an” and “the” include singular as well as plural references unless the context clearly dictates otherwise. For example, the term “a triglyceride” includes one or more triglycerides which may form together a lipid base or a lipid blend.

The term “consisting essentially of” is used to define the lipid composition which include the recited elements but exclude other elements, i.e., the term lipid composition is used to define a composition consisting essentially only lipids. “Consisting of” shall thus mean excluding more than trace elements of other elements. Embodiments defined by each of these transition terms are within the scope of this invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Further, all numerical values, e.g. when referring the amounts or ranges of the elements constituting the various lipid compositions herein are approximations which are varied (+) or (−) by up to 20%, at times by up to 10% of the stated values. It is to be understood, even if not always explicitly stated that all numerical designations are preceded by the term “about”.

It should be noted that where various embodiments are described by using a given range, the range is given as such merely for convenience and brevity and should not be construed as an inflexible limitation on the scope of the invention. Accordingly, the description of a range should be considered to have specifically disclosed all the possible sub-ranges as well as individual numerical values within that range.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

It is noted that features of certain embodiments of the invention which are described in detail in the context of one aspect of the invention, may be applicable in other aspects of the invention.

The invention will now be exemplified in the following description of experiments that are carried out in accordance with the invention. It is to be understood that these examples are intended to be in the nature of illustration rather than of limitation. Obviously, many modifications and variations of these examples are possible in light of the above teaching. It is therefore, to be understood that within the scope of the appended claims, the invention may be practiced otherwise, in a myriad of possible ways, than as specifically described herein below.

### DESCRIPTION OF NON-LIMITING EXAMPLES

In the present description as well as in the non-limiting examples provided below reference is made to fat bases and fat blends. It is to be understood that the term “fat base” or “fat concentrate” or “fat base concentrate” is used to denote the enzymatically prepared lipid composition comprising a mixture of vegetable-derived triglycerides with high sn-2 palmitic acid; while the term “fat blend” is used to denote a lipid composition comprising a fat base and a mixture of edible vegetable oil/s.

As shown below, the fat blend is a fat base comprising mainly triglycerides with high total palmitic and high sn-2 palmitic acid mixed with other edible vegetable oils. The edible vegetable oil/s may be natural vegetable oil/s, randomized vegetable oil/s, interesterified vegetable oils, enzymatically interesterified vegetable oils, at least two vegetable oils which were co-randomized, at least two vegetable oils which were co-interesterified. Generally, this fat blend is used as a fat fraction in infant formulas and can be used in other baby foods such as biscuits, bar, etc., food articles and clinical nutrition products.

As used herein, the term “palmitic acid sn-2 ratio” or “C16:0 sn-2 ratio” means the level of palmitic acid moieties at the sn-2 position of the glycerol backbone as % of total palmitic acid in the triglyceride composition (oil).

As used herein, the term “TG” refers to triglyceride.

### Example 1—Preparing Fat Bases and Fat Blends

Table 1 details the contents of several fat bases enriched with a high content of palmitic acid at the sn-2 position. The fat bases comprise a high percentage of palmitic acid, C16:0, at the sn-2 position of triacylglycerol (TAG), and high percentage of unsaturated fatty acids at the sn-1 and sn-3 positions.

The fat bases are prepared as described in publication WO 2005/036987 [3] which is fully incorporated herein by reference. Generally, a mixture of triglycerides, rich in palmitic acid (preferably above 78%) are reacted with a mixture of free fatty acids (FFA) rich in oleic acid (preferably above 75%), with a low content of palmitic and stearic acids (preferably below 6%).

Briefly, the triglyceride mixture may be produced from double-fractioned palm stearin and the FFA mixture is obtained from palm kernel oil after fractionation, or from high oleic sunflower oil. The two mixtures are blended in stirred (optionally large scale) reactors with no additional solvent. To this mixture is added a suitable lipase and the mixture of triglycerides, FFA and catalyst is stirred at 50° C.-60° C. for about 3-9 hours, to yield the final and desired triglycerides mixture. Any excess FFAs are removed.

The triglyceride product may be further treated in order to improve color, odor and taste with bleaching and deodorization stages. Optionally, the product is fortified with natural antioxidants to increase the shelf life of the product. The catalyst can be further recycled, to be re-used in further batches.

TABLE 1
Fat bases composition

| | Fat Base No. | | | | | | | | | | |
|---|---|---|---|---|---|---|---|---|---|---|---|
| fatty acid* | 1 | 2 | 3 | 4 | 5 | 6 | 7 | 8 | 9 | 10 | 11 |
| C16:0 | 32 | 29.4 | 29.6 | 32.6 | 32.2 | 30.6 | 29 | 29 | 30 | 33 | 30 |
| C16:0 at sn-2 | 67.2 | 59.7 | 61.3 | 66.1 | 66 | 62.9 | 53.9 | 55.6 | 59 | 52.9 | 55.8 |
| of total fatty | | | | | | | | | | | |
| acids at sn-2 | | | | | | | | | | | |
| Ratio (%) | 70.0 | 67.7 | 69.0 | 67.6 | 68.3 | 68.5 | 62 | 64 | 64 | 53.5 | 62 |
| of C16:0 at | | | | | | | | | | | |
| sn-2 out of | | | | | | | | | | | |
| total C16:0 | | | | | | | | | | | |
| C18:0 | 4 | 4.4 | 4.4 | 4 | 4.1 | 3.8 | 2.6 | 2.6 | 3 | 3 | 3 |
| C18:1 | 53.1 | 55.9 | 55.5 | 53.1 | 53.4 | 55 | 55.5 | 56 | 56.1 | 52 | 56.1 |
| C18:2 | 8 | 7.8 | 8.2 | 8 | 7.9 | 8.3 | 9 | 9 | 8.5 | 10 | 8.5 |

*All numbers represent % (w/w), meaning the weight % of the specific fatty acid out of total fatty acids weight, except for the term “C16:0 at sn-2” which represents the % (w/w) palmitic acid at sn-2 out of total sn-2 positioned fatty acids and the term “Ratio” which represents % of C16:0 at sn-2 position out of total C16:0 calculated by [(% of C16:0 at sn-2 out of total sn-2 positioned fatty acids)/3)/(% total C16:0)] × 100.

The fat bases are then used to form the fat blends which comprise also other oils. The fat base may represent from about 25% up to about 83% of the fat blends suitable for use in a formula for use in the invention. The blends comprising the fat bases of Table 1 in combination with other fats are provided in Table 2.

Specifically, Table 2 details the contents of blends comprising one of fat bases 1, 7, 8, 9, 10 or 11. The fat blends are prepared by blending the selected fat base with other oils (e.g., palm kernel oil, coconut oil, palm oil, sunflower oil, corn oil, safflower oil, rapeseed oil and soybean oil). As such, the fatty acids composition of the blends results from the fatty acids composition of both the fat base and of the other oils mixed with the fat base.

TABLE 2
Fat blends composition

| | Fat Blend No. | | | | | | | | | |
|---|---|---|---|---|---|---|---|---|---|---|
| | Fat | Fat | Fat | Fat | Fat | Fat | Fat | Fat | Fat | Fat |
| fat* | blend 1 | blend 2 | blend 3 | blend 4 | blend 5 | blend 6 | blend 7 | blend 8 | blend 9 | blend 10 |
| C12:0 | 11.1 | 7.2 | 7.8 | 6.5 | 4.4 | 8.14 | 8.7 | 13.4 | 10.4 | 10 |
| C14:0 | 4.5 | 3.1 | 3.3 | 2.8 | 2.1 | 2.94 | 3.54 | 5.3 | 4.3 | 4.2 |
| C16:0 | 22.8 | 25.4 | 26.9 | 25.1 | 27.7 | 21.60 | 20.99 | 15 | 22.3 | 17 |
| C16:0 at sn-2 | 33.4 | 42.9 | 48.9 | 50.8 | 56.9 | 31.3 | 31.8 | 25 | 28.8 | 16 |
| of total fatty | | | | | | | | | | |
| acids at sn-2 | | | | | | | | | | |
| Ratio (%) | 48.7 | 56.3 | 60.7 | 67.4 | 68.5 | 48.31 | 50.46 | 55 | 43 | 31.5 |
| sn-2 C16:0 of | | | | | | | | | | |
| total C16:0 | | | | | | | | | | |
| C18:0 | 2.3 | 3.0 | 3.1 | 3.5 | 4.0 | 2.65 | 2.65 | 2.9 | 4.4 | 3.2 |
| C18:1 | 38.4 | 40.8 | 41.6 | 47.9 | 46.6 | 42.71 | 44.37 | 39.7 | 38.5 | 41.7 |
| C18:2 | 13.5 | 15.6 | 12.8 | 8.6 | 11.7 | 17.96 | 16.43 | 15.3 | 14.0 | 18.2 |
| C18:3 | 1.7 | 0.6 | | 1.4 | | 1.69 | 1.52 | 2 | 1.5 | 2.1 |

*All numbers represent % (w/w), meaning the weight % of the specific fatty acid out of total fatty acids weight, except for: the term “C16:0 at sn-2” which represents the % (w/w) palmitic acid at sn-2 out of total sn-2 positioned fatty acids and the term “Ratio” which represents % of C16:0 at sn-2 position out of total C16:0 calculated by [(% of C16:0 at sn-2 out of total sn-2 positioned fatty acids)/3)/(% total C16:0)] × 100.

Table 3 details the content of specific Fat blend 11.

TABLE 3
Fat blend 11 composition (with 30% fat base)

| Fatty acid | % of fatty acids* |
|---|---|
| C10:0 | 1.3 |
| C12:0 | 10.3 |
| C14:0 | 4.3 |
| C16:0 | 23.5 |
| C16:0 at sn-2 of total fatty acids at sn-2 | 30.3 |
| Ratio (%) of C16:0 at sn-2 of total C16:0 | 43 |
| C18:0 | 3.2 |
| C18:1 | 39.2 |
| C18:2 | 13.6 |
| C18:3 | 1.7 |
| C20:0 | 0.3 |
| C20:1 | 0.3 |
| C22:0 | 0.2 |
| % fat base in fat blend 11 | 30 |

*All numbers represent % (w/w), meaning the weight % of the specific fatty acid out of total fatty acids weight, except for: the term “C16:0 at sn-2” which represents the % (w/w) palmitic acid at sn-2 out of total sn-2 positioned fatty acids and the term “Ratio” which represents % of C16:0 at sn-2 position out of total C16:0 calculated by [(% of C16:0 at sn-2 out of total sn-2 positioned fatty acids)/3)/(% total C16:0)] × 100.

Table 4 details the content of specific Fat blend 12.

TABLE 4
Fat Blend 12 composition (with 43% fat base)

| | % from total |
|---|---|
| Fatty acid | Fatty acids* |
| C8:0 | 1.6 |
| C10:0 | 1.5 |
| C12:0 | 10.6 |
| C14:0 | 3.9 |
| C16:0 | 17.2 |
| C16:0 at sn-2 of total fatty acids at sn-2 | 26.3 |
| Ratio (%) of sn-2 palmitic acid of total palmitic acid | 51 |
| C18:0 | 2.4 |
| C18:1 | 41.1 |
| C18:2 | 18.2 |
| C18:3 | 2.2 |

*All numbers represent % (w/w), meaning the weight % of the specific fatty acid out of total fatty acids weight, except for: the term “C16:0 at sn-2” which represents the % (w/w) palmitic acid at sn-2 out of total sn-2 positioned fatty acids and the term “Ratio” which represents % of C16:0 at sn-2 position out of total C16:0 calculated by [(% of C16:0 at sn-2 out of total sn-2 positioned fatty acids)/3)/(% total C16:0)] × 100.

Table 5 details the content of specific Fat blend 13.

TABLE 5
Fat Blend 13 composition

| | | % (w/w) | | | % (w/w) |
|---|---|---|---|---|---|
| | % (w/w) | on sn-2 | sn-2* | sn-1/3** | on sn-1/3 |
| | on TG | position | ratio | ratio | position |
| C12 | 0.2 | 0.3 | 50.0 | 50.0 | 0.2 |
| C14 | 15.0 | 21.0 | 46.7 | 53.3 | 12.0 |
| C16 | 17.0 | 23.0 | 45.1 | 54.9 | 14.0 |
| C18 | 13.0 | 24.0 | 61.5 | 38.5 | 7.5 |
| C18:1n9 | 47.0 | 30.0 | 21.3 | 78.7 | 55.5 |
| C18:2n6 | 7.4 | 1.0 | 4.5 | 95.5 | 10.6 |
| C18:3n3 | 0.2 | 0.2 | 33.3 | 66.7 | 0.2 |
| saturated | 45.2 | 68.3 | | | 33.7 |
| unsaturated | 54.6 | 31.2 | | | 66.3 |
| palmitic of | 37.6 | 33.7 | | | 41.6 |
| saturated | | | | | |
| oleic of | 86.1 | 96.2 | | | 83.7 |
| unsaturated | | | | | |
| linoleic of | 13.6 | 3.2 | | | 16.0 |
| unsaturated | | | | | |
| linolenic of | 0.4 | 0.6 | | | 0.3 |
| unsaturated | | | | | |

% (w/w) on TG represents % of the specific fatty acid out of total fatty acids.
% (w/w) on sn-2 position represents the % of the specific fatty acid at sn-2 position out of total fatty acids at sn-2 position.
*sn-2 ratio represents the ratio between the amount of certain fatty acid moiety at the sn-2 position of the glycerol backbone out of total amount of said fatty acid in the triglyceride composition.
**sn-1/3 ratio represents the ratio between the amount of certain fatty acid moiety at the sn-1 and sn-3 positions of the glycerol backbone out of total amount of said fatty acid in the triglyceride composition.
% (w/w) on sn-1/3 position represents the % of the specific fatty acid at sn-1 and sn-3 positions of the glycerol backbone out of total fatty acids at sn-1 and sn-3 positions of the glycerol backbone.
For example, C16:0 sn2 ratio is calculated by: [(% of C16:0 at sn-2 out of total sn-2 positioned fatty acids)/3)/(% total C16:0)] × 100.

Table 6 details the content of specific Fat blend 14.

TABLE 6
Fat Blend 14 composition

| | | % (w/w) | | | % (w/w) |
|---|---|---|---|---|---|
| | % (w/w) | on sn-2 | sn-2* | sn-1/3** | on sn-1/3 |
| | on TG | position | ratio | ratio | position |
| C12 | 0.2 | 0.3 | 50.0 | 50.0 | 0.2 |
| C14 | 15.0 | 30.0 | 66.7 | 33.3 | 7.5 |
| C16 | 20.0 | 30.0 | 50.0 | 50.0 | 15.0 |
| C18 | 5.9 | 3.9 | 22.0 | 78.0 | 6.9 |
| C18:1n9 | 47.1 | 24.2 | 17.1 | 82.9 | 58.6 |
| C18:2n6 | 7.4 | 4.9 | 22.1 | 77.9 | 8.7 |
| C18:3n3 | 0.2 | 0.2 | 33.3 | 66.7 | 0.2 |
| saturated | 41.1 | 64.2 | | | 29.6 |
| unsaturated | 54.7 | 29.3 | | | 67.4 |
| palmitic of | 48.7 | 46.7 | | | 50.8 |
| saturated | | | | | |
| oleic of | 86.1 | 82.6 | | | 86.9 |
| unsaturated | | | | | |
| linoleic of | 13.5 | 16.7 | | | 12.8 |
| unsaturated | | | | | |
| linolenic of | 0.4 | 0.7 | | | 0.3 |
| unsaturated | | | | | |

% (w/w) on TG represents % of the specific fatty acid out of total fatty acids.
% (w/w) on sn-2 position represents the % of the specific fatty acid at sn-2 position out of total fatty acids at sn-2 position.
*sn-2 ratio represents the ratio between the amount of certain fatty acid moiety at the sn-2 position of the glycerol backbone out of total amount of said fatty acid in the triglyceride composition.
**sn-1/3 ratio represents the ratio between the amount of certain fatty acid moiety at the sn-1 and sn-3 positions of the glycerol backbone out of total amount of said fatty acid in the triglyceride composition.
% (w/w) on sn-1/3 position represents the % of the specific fatty acid at sn-1/3 positions of the glycerol backbone out of total fatty acids at sn-1 and sn-3 positions of the glycerol backbone.
For example, C16:0 sn2 ratio is calculated by: [(% of C16:0 at sn-2 out of total sn-2 positioned fatty acids)/3)/(% total C16:0)] × 100.

### Example 2—Effect of Palmitic Acid on Catch Up Growth in Young Rats

The aim of this study was to examine whether the lipid composition of the present invention affects the growth process and especially the efficiency of the catch up (CU) growth process. An animal model of food restriction followed by ad libidum feeding for enabling catch up growth was used.

### Experimental Set Up:

Experiments were performed on pre-pubertal 23 day-old male Sprague-Dawley rats of average weight 50 g (Harlan Laboratories Ltd., Jerusalem, Israel). All animals were kept under the same experimental conditions: mean ambient temperature 22±1° C., mean relative humidity 50±2%, 12 h light/dark cycle; lights off at 18:00 h. All had free access to water, and all were fed the same commercial rat chow during the food-restriction period (Teklad Rodent Diet (2018SC), 3.4 kcal/g; Teklad, South Easton, Mass., USA). Rats were kept separately in single cages. Animals were observed daily.

All experiments were approved by the Institutional Animal Care and Use Committee.

Animals were fed one of the special diets that differed in the ratio of palmitic acid at sn-2 [12.9% in the Control Diet (CD) vs. 53% in the diet according to the invention which is referred to herein as Test Diet (TD)] while having similar levels of total palmitic acid and other fatty acids (see Table 7 for detailed fatty acid composition). All other ingredients (corn starch, sucrose, cellulose, oil, vitamin and minerals including calcium carbonate and calcium phosphate) were identical in the TD and CD diets.

A preliminary study was performed to check if the diets were well tolerated by the rats. For this purpose, two groups of Sprague-Dawley rats (n=10) were housed individually in separate cages to allow careful monitoring of the amount of food consumed and were allowed to eat one of the two different diets ad libitum, with no restriction, for 30 days.

Body weight was measured every other day and food consumption was measured daily. At the age of 23 days, after two days of acclimatization to the solitary cages, rats were allowed a restricted feeding protocol of 60% of the normal daily intake of the commercial regular chow for 17 days. The 40% restriction was calculated on the basis of previous studies wherein animals were housed individually and the amount of food consumed each day was measured together with the animal's weight and weight gain. On day 17 they were divided into two groups of unrestricted re-feeding for 9 days with one of two types of iso-caloric, iso-protein diets.

Table 7 details the fat composition of the control diet (CD) and the fat composition of the test diet (TD) according to the invention.

TABLE 7
The fat composition provided in a control diet and in a diet according to the present invention

| | Control Diet (CD) | | | | | Test Diet (TD) | | | | |
|---|---|---|---|---|---|---|---|---|---|---|
| | | % (w/w) | | | % (w/w) | | % (w/w) | | | % (w/w) |
| | % (w/w) | on sn-2 | sn-2* | sn-1/3** | on sn-1/3 | % (w/w) | on sn-2 | sn-2* | sn-1/3** | on sn-1/3 |
| | on TG | position | ratio | ratio | position | on TG | position | ratio | ratio | position |
| C14 | 0.3 | 0.2 | 24.7 | 75.3 | 0.4 | 0.2 | 0.3 | 59.3 | 40.7 | 0.1 |
| C16 | 14.4 | 5.6 | 12.9 | 87.1 | 18.8 | 13.9 | 22.0 | 53.0 | 47.0 | 9.8 |
| C16:1 | 0.2 | 0.1 | 22.2 | 77.8 | 0.2 | 0.1 | 0.1 | 23.3 | 76.7 | 0.1 |
| C18 | 3.1 | 0.6 | 6.2 | 93.8 | 4.4 | 3.4 | 1.2 | 11.4 | 88.6 | 4.5 |
| C18:1n9 | 32.1 | 36.3 | 37.7 | 62.3 | 30.0 | 36.7 | 27.2 | 24.7 | 75.3 | 41.4 |
| C18:1n11 | 1.1 | 0.5 | 14.5 | 85.5 | 1.4 | 1.0 | 0.4 | 13.3 | 86.7 | 1.2 |
| C18:2n6 | 37.9 | 52.9 | 46.6 | 53.4 | 30.4 | 34.2 | 45.6 | 44.4 | 55.6 | 28.5 |
| C18:3n3 | 1.6 | 2.7 | 58.1 | 41.9 | 1.0 | 1.3 | 2.2 | 59.0 | 41.0 | 0.8 |
| SAT | 17.8 | 6.4 | | | 23.5 | 17.4 | 23.5 | | | 14.4 |
| Un-SAT | 72.8 | 92.5 | | | 62.9 | 73.2 | 75.5 | | | 72.1 |
| C16 of | 80.8 | 87.3 | | | 80.0 | 79.6 | 93.7 | | | 68.0 |
| SAT | | | | | | | | | | |
| C18:1 of | 44.1 | 39.3 | | | 47.7 | 50.1 | 36.0 | | | 57.5 |
| Un-SAT | | | | | | | | | | |
| C18:2 of | 52.1 | 57.2 | | | 48.3 | 46.7 | 60.4 | | | 39.6 |
| Un-SAT | | | | | | | | | | |
| C18:3 of | 2.1 | 2.9 | | | 1.6 | 1.7 | 3.0 | | | 1.1 |
| Un-SAT | | | | | | | | | | |

% (w/w) on TG represents % of the specific fatty acid out of total fatty acids.
% (w/w) on sn-2 position represents the % of the specific fatty acid at sn-2 position out of total fatty acids at sn-2 position.
*sn-2 ratio represents the ratio between the amount of certain fatty acid moiety at the sn-2 position of the glycerol backbone out of total amount of said fatty acid in the triglyceride composition.
**sn-1/3 ratio represents the ratio between the amount of certain fatty acid moiety at the sn-1 and sn-3 positions of the glycerol backbone out of total amount of said fatty acid in the triglyceride composition.
% (w/w) on sn-1/3 position represents the % of the specific fatty acid at sn-1/3 positions of the glycerol backbone out of total fatty acids at sn-1 and sn-3 positions of the glycerol backbone.
For example, C16:0 sn2 ratio is calculated by: [(% of C16:0 at sn-2 out of total sn-2 positioned fatty acids)/3)/(% total C16:0)] × 100.
SAT refers to “saturated fatty acids”.

Un-SAT refers to “unsaturated fatty acids”.

The animals were fed for 9 days and then sacrificed by CO₂ inhalation. At sacrifice, blood was collected by cardiac puncture; serum was separated and kept at −20° C. until analyzed. Internal organs were removed, weight and stored at −70° C. Humeral bones were manually cleaned of the soft tissue, measured using a digital caliper, and prepared for further analysis. Liver, bone and growth plates were removed, weight, measured, and quickly frozen in liquid nitrogen and then kept in −70° C.

Tibiae and humeri of each animal were carefully removed, cleaned, and measured for length with a digital caliper. Bones were fixed in 4% neutral buffered formalin for 48 hours at room temperature, decalcified with EDTA and HCl (Calci-Clear Rapid, cat. no. HS-105, National Diagnostics, Atlanta, Ga.) for 7 hours, dehydrated with a graded ethanol series (70%, 95%, 100%), and stabilized by two sequential changes of chloroform for paraffin embedding. Histological studies of de-paraffinized sections stained with hematoxylin-eosin and Alcian blue were used for general morphological evaluation using a Digital camera with appropriate morphometric software (Olympus Optical Co. GmbH, Hamburg, Germany). Epiphyseal growth plate height measurements (from the reserve zone to the ossification front of the metaphyseal bone), were performed on stained paraffin sections of 5 μm thickness photographed under the Olympus BX40 microscope using a Olympus DP71 camera and analyzed using the Image-Pro software (version 4.5.1.22, Media Cybernetics, Inc, Rockville, Md., USA).

To study changes in gene expression due to the changes in diet, total RNA was extracted from the growth plate and internal organs and analyzed by Affymetrix expression array. Total RNA was extracted from liver and EGP tissues of CD and TD groups with miReasy Mini Kit (Qiagen, Valencia, Calif., USA) according to the manufacturer's protocol (n=4 per each group). The quantity and quality of the RNA were evaluated using a Nanodrop spectrophotometer (Nanodrop Ltd, USA) and Bioanlayzer (Agilent-2100 Bioanalyzer) with values of: A260/A280>2.0 and A260/A230>1.7 and RNA integrity number (RIN) value >6.1. Equal amounts of RNA from all groups were used for analysis by the Rat Affymetrix gene chip expression array (Affymetrix; Rat Gene 2.X ST). Quality control approved all samples. Clustering and ANOVA calculations were done using the Partek Genomics Suite, v 6.6.

Affymetrix expression arrays are quartz chips called GeneChips, which enable researchers to scan simultaneously the expression of thousands of genes in a biological sample (for example of use of Affymetrix for growth plate analysis see [7] and [8]).

### Results:

Table 8 details the growth parameters that were tested and the results obtained.

TABLE 8
Growth parameters of rats consuming a diet comprising
a lipid composition according to the invention (TD)
in comparison with rats consuming a control diet (CD)

| | Control Diet (CD) | Test Diet (TD) |
|---|---|---|
| Diet | n = 7 | n = 6 |
| Weight (gr) | 131.3 ± 7.9 | 135.6 ± 8.4 |
| Weight gain (gr) | 83.5 ± 5.1 | 87.9 ± 6.4 |
| Liver weight (gr) | 6.9 ± 0.3 | 6.8 ± 0.5 |
| Liver Fat Content (mg/100 mg tissue) | 4.5 ± 0.5 | 4.7 ± 0.7 |
| Full Humerus Length (mm) | 19.86 ± 0.5 | 20.4 ± 0.3 |
| EGP height (mm) | 0.39 ± 0.02 | 0.41 ± 0.02 |
| Proliferative part (mm) | 0.22 ± 0.04 | 0.21 ± 0.03 |
| Hypertrophic part (mm) | 0.18 ± 0.02 | 0.2 ± 0.02 |
| Proliferative/Hypertrophic ratio | 1.21 ± 0.18 | 1.03 ± 0.14 |
| (at EGP) | | |
| IGF-I (ng/ml) | 868.1 ± 193.7 | 894.2 ± 108.1 |
| Leptin (pg/ml) | 1461 ± 196 | 1448 ± 434.8 |

The preliminary experiment showed that both diets were very well tolerated by the rats; there were no adverse effects to any of the diets.

1. Body weight and weight gain of the catch up model animals was higher in the group of animals fed the diet with the lipid composition of the invention (TD vs CD). The liver weight and liver fat content were similar between the groups.

2. After the re-feeding period, serum IGF-1 level was higher and leptin level was lower in the group of animals fed the diet with the lipid composition of the invention (TD vs CD).

3. The CD and TD groups differed in several parameters:
a. The length of the humeri was shorter in the CD group compared to the rats fed TD diet.
b. EGP height was greater in the TD fed animals compared to those fed CD, suggesting better growth potential.
c. The increase in the EGP height was due to an increase in both proliferative and hypertrophic zones of the EGP; with the differences in the hypertrophic zone significantly higher in the TD group compared to the CD group. Without wishing to be bound by theory, the ratio between the proliferative/hypertrophic zones in the EGP suggests that the effect was most pronounced in the proliferative zone; however, the morphology of the EGP was intact, indicating that there were no adverse effects to any of the diets.

4. Affymetrix analysis performed on RNA samples from the liver and the EGP demonstrate regulation of the expression of genes related to growth within the group administrated with TD in comparison with the CD group:
a. 112 metabolic gene were significantly different between the two study groups.
b. Nampt, Alas1 and Mllt3 genes were significantly increased by >1.5 fold (TD>CD).
c. Early growth response gene 1 (EGR-1), a transcription factor related to growth down-regulation, was down regulated by the lipid of the invention.
d. Some cartilage specific genes were affected being highly expressed in TD samples compared to CD.

### Conclusion:

The results above demonstrate the benefit of the lipid composition of the present invention on growth parameters especially during catch up growth. Animals fed with a diet comprising the lipid composition of the present invention had longer bones and higher growth plates (EGP) in the bones in comparison with animals fed with the control diet. The increase in bone length and EGP height with a good quality of the bone significantly point to the beneficial effect of the lipid of the invention on linear growth, as higher, yet intact EGP indicates a better growth potential. In addition, changes in gene expression supported growth promotion following consumption of a composition according to the invention in comparison with the control.

All those point to an effect of the lipid of the invention toward a final higher stature of the subject due to the increased growth potential.

### Example 3—The Effect of Infant Formula with Different Fat Components on Growth in Formula-Fed Infants Small for Gestational Age

The effect of the fat component in the infant formula on infant growth is examined in a double blind randomized clinical trial in human term formula fed infants.

### Study Design

Healthy term infants (born at GA≥37) which are small for gestational age and younger than 14 days are eligible for entry to this multi-center, randomized, double-blind study. Infants are excluded from the study if they suffer from a congenital or chromosomal disorder, neonatal morbidities, or metabolic illnesses.

Infants are randomly assigned to one of the two groups:

Group A: Infants fed with infant formula A (see Table 9 and Table 10) containing a lipid composition according to the present invention.

Group B: Infants fed with a control formula B (see Table 9 and Table 10).

Both formulas are produced by the same manufacturer and under the same conditions, using identical ingredients from the same batches except the fat ingredient, which differed mainly in the fatty acid structural distribution.

The study is conducted according to the principles of the Declaration of Helsinki and good clinical practices. The protocol is approved by the Ethics Committees of clinical centers; all the parents give written informed consent prior to inclusion.

Table 9 below details the composition of Formula A (according to the present invention) and Formula B (control).

TABLE 9
Composition of study formulas (per 100 gram powder)

| | Formula A (per | Formula B (per |
|---|---|---|
| | 100 gr powder) | 100 gr powder) |
| | (according to the invention) | (control) |
| Energy (kcal) | 502 | 498 |
| Protein (g) | 11.1 | 10.9 |
| Carbohydrate (g) | 56.8 | 57.8 |
| Fat (g) | 25 | 24.5 |

Table 10 below details the fatty acids composition of Formula A (according to the present invention) and Formula B (control).

TABLE 10
Fatty acids Composition of Study Formulas (%)

| | Formula A | | | | | Formula B | | | | |
|---|---|---|---|---|---|---|---|---|---|---|
| | | % (w/w) | | | % (w/w) | | % (w/w) | | | % (w/w) |
| | % (w/w) | on sn-2 | sn-2* | sn-1/3** | sn-1/3 | % (w/w) | on sn-2 | sn-2* | sn-1/3** | sn-1/3 |
| | on TG | position | ratio | ratio | position | on TG | position | ratio | ratio | position |
| C8 | 0.7 | 0.2 | | | | 0.7 | 0.3 | | | |
| C10 | 0.7 | 0.3 | | | | 0.7 | 0.4 | | | |
| C12 | 9.1 | 9.9 | | | | 9.4 | 11.4 | | | |
| C14 | 3.3 | 3.3 | 33.3 | 66.7 | 3.3 | 3.5 | 3.6 | 34.3 | 65.7 | 3.5 |
| C16 | 20.3 | 26.7 | 43.8 | 56.2 | 17.1 | 20.8 | 7.6 | 12.2 | 87.8 | 27.4 |
| C18 | 3.6 | 1.4 | 13.0 | 87.0 | 4.7 | 3.3 | 0.6 | 6.1 | 93.9 | 4.7 |
| C18:1n9 | 42.8 | 34.1 | 26.6 | 73.4 | 47.2 | 42.3 | 49.8 | 39.2 | 60.8 | 38.6 |
| C18:2n6 | 16.2 | 21.0 | 43.2 | 56.8 | 13.8 | 16.0 | 23.3 | 48.5 | 51.5 | 12.4 |
| C18:3n3 | 1.9 | 3.2 | 56.1 | 43.9 | 1.3 | 1.7 | 2.9 | 56.9 | 43.1 | 1.1 |
| SAT | 37.7 | 41.8 | | | 25.1 | 38.4 | 23.9 | | | 35.5 |
| Un-SAT | 60.9 | 58.3 | | | 62.2 | 60.0 | 76.0 | | | 52.0 |
| C16 of | 53.8 | 63.9 | | | 68.1 | 53.8 | 63.9 | | | 77.2 |
| SAT | | | | | | | | | | |
| C18:1 of | 70.3 | 58.5 | | | 75.8 | 70.3 | 58.5 | | | 74.1 |
| Un-SAT | | | | | | | | | | |
| C18:2 of | 26.6 | 36.0 | | | 22.2 | 26.6 | 36.0 | | | 23.8 |
| Un-SAT | | | | | | | | | | |
| C18:3 of | 3.1 | 5.5 | | | 2.0 | 3.1 | 5.5 | | | 2.1 |
| Un-SAT | | | | | | | | | | |

% (w/w) on TG represents % of the specific fatty acid out of total fatty acids.
% (w/w) on sn-2 position represents the % of the specific fatty acid at sn-2 position out of total fatty acids at sn-2 position.
*sn-2 ratio represents the ratio between the amount of certain fatty acid moiety at the sn-2 position of the glycerol backbone out of total amount of said fatty acid in the triglyceride composition.
**sn-1/3 ratio represents the ratio between the amount of certain fatty acid moiety at the sn-1 and sn-3 positions of the glycerol backbone out of total amount of said fatty acid in the triglyceride composition.
% (w/w) on sn-1/3 position represents the % of the specific fatty acid at sn-1 and sn-3 positions of the glycerol backbone out of total fatty acids at sn-1 and sn-3 positions of the glycerol backbone.
For example, C16:0 sn2 ratio is calculated by: [(% of C16:0 at sn-2 out of total sn-2 positioned fatty acids)/3)/(% total C16:0)] × 100,
SAT refers to “saturated fatty acids”.
Un-SAT refers to “unsaturated fatty acids”.

Infant growth parameters are followed during intervention period as well as up to age of 6 years. The length/height is measured every month during first 6 months of life, then every 2 months until age of 1 year, every 6 months until age of 3 and once a year until age of 6 and once every two years until age of 18. The length/height is compared to norms according to WHO growth charts and the percentile is calculated compared to the norms. Additionally at the end of the study a skeletal age assessment is performed.

### Conclusion

Infant formulas comprising a composition according to the present invention have a positive effect on growth patterns and on longer growth period (thus may increase growth potential).

### Example 4—The Effect of a Formula with Different Fat Components on Growth in Children Short Compared to Norms

The effect of the fat component in a formula on children growth is examined in a double blind randomized clinical trial in children with height below 25^{th} percentile compared to norms for their age.

### Study Design

Healthy children aged 5-10 years with height below 25th percentile compared to norms for their age are eligible for entry to this multi-center, randomized, double-blind study. Children are excluded from the study if they suffer from any metabolic or gastrointestinal disease, genetic disorders or take any chronic medical treatment.

Children are randomly assigned to one of the two groups:

Group A: children fed with formula A (see Table 11) containing a lipid composition according to the present invention.

Group B: children fed with formula B (see Table 11) with similar fatty acids composition but differ in its triglyceride positioning.

All formulas are produced by the same manufacturer and under the same conditions, using identical ingredients from the same batches except the fat ingredient, which differed mainly in the fatty acid structural distribution.

The study is conducted according to the principles of the Declaration of Helsinki and good clinical practices. The protocol is approved by the Ethics Committees of clinical centers; all the parents gave written informed consent prior to inclusion.

Table 11 below details the fatty acids composition of Formula A (according to the present invention) and Formula B (control).

TABLE 11
Fatty acids Composition of Study Formulas (%)

| | |
|---|---|
| Formula A | Formula B |

| | % (w/w) | sn-2* | % (w/w) | sn-2* |
|---|---|---|---|---|
| | on TG | ratio | on TG | ratio |
| C8:0 | 0.7 | 0.0 | 0.7 | 0.0 |
| C10:0 | 0.7 | 19.0 | 0.7 | 9.5 |
| C12:0 | 9.1 | 43.2 | 9.4 | 41.5 |
| C14:0 | 3.3 | 40.4 | 3.5 | 40.0 |
| C16:0 | 20.3 | 44.3 | 20.8 | 13.1 |
| C18:0 | 3.6 | 13.9 | 3.3 | 7.1 |
| C18:1 | 42.8 | 25.2 | 42.3 | 38.5 |
| C18:2 | 16.2 | 40.1 | 16.0 | 48.3 |
| C18:3 | 1.9 | 54.4 | 1.7 | 56.9 |

% (w/w) on TG represents % of the specific fatty acid out of total fatty acids.
*sn-2 ratio represents the ratio between the amount of certain fatty acid moiety at the sn-2 position of the glycerol backbone out of total amount of said fatty acid in the triglyceride composition.

Children growth parameters are followed during 12 months of intervention period and for 3 additional years. The height is measured every month during intervention period and 1 year from the end of the intervention. The height is compared to norms according to WHO growth charts and the percentile is calculated compared to the norms. Additionally, at the end of the study a skeletal age assessment is performed.

### CONCLUSION

The formula comprising a composition according to the present invention has a positive effect on growth patterns and on growth period (thus may increase growth potential).

1. A method for one or both of improvising growth in a subject and treating at least one growth problem in a subject, said method comprising administering to said subject an edible lipid composition comprising a vegetable-derived fat source, wherein said fat source is a triglyceride fat source comprises triglycerides with 13-55% palmitic acid moieties out of the total fatty acids and wherein the level of palmitic acid moieties at the sn-2 position of the glycerol backbone is at least 30% of total palmitic acid.

2. The method according to claim 1 wherein at least about 20% of the total fatty acid moieties at the sn-2 position of the glycerol backbone are saturated.

3. The method according to claim 1 wherein at least about 50% of the total fatty acid moieties at the sn-1 and sn-3 positions of the glycerol backbone are unsaturated.

4. The method according to claim 1 wherein at least about 40% of the fatty acids are unsaturated fatty acid moieties.

5. The method according to claim 1 wherein at least about 50% of the unsaturated fatty acids are oleic acid.

6. The method according to claim 1 wherein at least about 15% of the unsaturated fatty acids are linoleic acid.

7. The method according to claim 1 wherein at most about 5% of the unsaturated fatty acids are linolenic acid.

8. The method according to claim 1 wherein at least about 35% of the unsaturated fatty acid moieties at the sn-1 and sn-3 positions are oleic acid moieties.

9. The method according to claim 1 wherein at least about 4% of the unsaturated fatty acid moieties at the sn-1 and sn-3 positions are linoleic acid moieties.

10. The method according to claim 1 wherein said subject is an infant, a toddler, a child or an adolescent.

11. (canceled)

12. (canceled)

13. (canceled)

14. (canceled)

15. The method according to claim 1, wherein said subject is a non-healthy subject.

16. The method according to claim 1, wherein said method is for improving growth.

17. The method according to claim 1, wherein said method is for maximizing said subject's growth potential.

18. The method of claim 1 wherein said method is for facilitating catch up growth, for increasing bone length, for increasing growth plates height, for increasing growth-related gene expression or for treating at least one growth problem.

19. (canceled)

20. (canceled)

21. (canceled)

22. (canceled)

23. The method according to claim 14 wherein said growth problem is a reduced growth rate, a failure to thrive, an endocrine disease, Turner syndrome or a chronic disease.

24. (canceled)

25. (canceled)

26. (canceled)

27. (canceled)

28. The method according to claim 1 wherein said subject is suffering from a growth problem.

29. The method according to claim 1 wherein said subject is at risk of developing at least one growth problem.

30. A method for one or both of improvising growth in a subject and treating at least one growth problem in a subject, said method comprising administering to said subject an edible vegetable-derived fat source, wherein said fat source is a triglyceride fat source comprising triglycerides with 13-55% palmitic acid moieties out of the total fatty acids and wherein the level of palmitic acid moieties at the sn-2 position of the glycerol backbone is at least 30% of total palmitic acid.

31. A method for one or both of improving growth in a subject and treating at least one growth problem in a subject, said method comprising administering to said subject a formula comprising the fat source according to claim 30.

32. The method of claim 31, wherein said formula is an infant formula, a toddler formula, a child formula or an adolescent formula.

33. (canceled)

34. (canceled)

35. (canceled)

36. A method for one or both of improving growth in a subject and treating at least one growth problem in a subject, said method comprising administering to said subject a formula comprising the composition according to claim 1

37. The method of claim 36, wherein said formula is an infant formula, a toddler formula, a child formula or an adolescent formula.
